(19) United States
(12) Patent Application Publication
(10) Pub. No.: US 2023/0237649 A1
(43) Pub. Date: Jul. 27, 2023

(54) Systems and Methods for Quantification of Liver Fibrosis with MRI and Deep Learning

(71) Applicant: CHILDREN'S HOSPITAL MEDICAL CENTER, Cincinnati, OH (US)

(72) Inventors: Jonathan DILLMAN, Cincinnati, OH (US); Lili HE, Mason, OH (US); Hailong LI, Cincinnati, OH (US)

(73) Assignee: CHILDREN'S HOSPITAL MEDICAL CENTER, Cincinnati, OH (US)

(21) Appl. No.: 17/919,030

(22) PCT Filed: Apr. 15, 2021
(86) PCT No.: PCT/US21/27398

§ 371 Date: Oct. 14, 2022

Related U.S. Application Data
(60) Provisional application No. 63/010,116, filed on Apr. 15, 2020.

(51) Int. Cl. G06T 7/00; A61B 5/00

(52) U.S. Cl. CPC G06T 7/0012; A61B 5/7264; G06T 2207/20084

(57) ABSTRACT

Embodiments provide a deep learning framework to accurately segment liver and spleen using a convolutional neural network with both short and long residual connections to extract their radiomic and deep features from multiparametric MRI. Embodiments will provide an “ensemble” deep learning model to quantify biopsy derived liver fibrosis stage and percentage using the integration of multiparametric MRI radiomic and deep features, MRE data, as well as routinely available clinical data. Embodiments will provide a deep learning model to quantify MRE-derived liver stiffness using multiparametric MRI, radiomic and deep features and routinely-available clinical data.

### CROSS-REFERENCE TO RELATED APPLICATIONS

The current application claims priority to U.S. Provisional Application Ser. No. 63/010,116, filed Apr. 15, 2020, the entire disclosure of which is incorporated by reference.

### BACKGROUND

Chronic liver diseases (CLD) are a common source of morbidity and mortality in both children and adults the United States and around the world.^{179, 180} Compared to other chronic diseases, CLD is associated with increased rates of hospitalization, longer hospital stays, and more frequent readmissions. CLD is responsible for large healthcare expenditures. A recent estimate (2017) of the lifetime costs of fatty liver disease in the U.S. was ˜$222 billion. Liver fibrosis (LF) is the most important and only histologic feature known to predict outcomes from CLD, with evaluation necessary for accurate staging as well as medical and surgical decision-making. The current standard for assessing LF is biopsy, which is costly, prone to sampling error, and invasive with poor patient acceptance. Thus, there is an urgent unmet need for noninvasive, highly accurate, and precise diagnostic technologies for detection and quantification of LF.

Detection and progression of such liver diseases is typically assessed using a combination of clinical history, physical examination, laboratory testing, biopsy with histopathologic assessment, and imaging.¹⁸¹ Historically, imaging assessment of chronic liver diseases has relied upon subjective assessment of liver morphology, echogenicity and echotexture on ultrasound, signal intensity at MRI, and appearances following intravenous contrast material administration at MRI and CT. However, recently there are increasingly available preclinical and clinical quantitative methods.¹⁸²⁻¹⁸⁵

In practice, radiologists most often use subjective visual liver assessments and less often MR elastography (MRE)-derived liver stiffness (LS) to suggest the presence and degree of LF. Visual assessment is qualitative, insensitive to early LF when CLD can be halted or reversed, and it has important research limitations. Deep learning (DL) can automatically, quantitatively and objectively recognize discriminative high-throughput imaging features that have potential to unveil early disease characteristics that are undetectable by the human eye. Application of DL to MRI may allow clinicians to more accurately detect and follow CLD by 1) quantifying LS from conventional imaging without the need for MRE; and, more importantly, 2) predicting histologic LF stage without the need for biopsy, while avoiding variability, reducing radiologist workload, and potentially reducing healthcare costs.

Elasticity imaging can be performed using either commercially-available ultrasound or MRI equipment and allows quantitative evaluation of liver stiffness. While liver stiffness can be impacted by a variety of physiologic and histopathologic processes, including inflammation,^{186,187} steatosis,¹⁸⁸ and passive congestion,^{189,190} liver stiffening is most often the result of tissue fibrosis in the setting of chronic liver diseases.^{186,191} MR elastography (MRE), in particular, uses an active-passive driver system (with the passive paddle placed over the right upper quadrant of the abdomen at the level of the costal margin) to create transverse (shear) waves in the liver. The displacement of liver tissue related to these waves can be imaged using a modified phase-contrast pulse sequence and can be used to create an elastogram (map or parametric image) of liver stiffness.^{192,193} Although MRE obviates the need for liver biopsy for some patients and allows more frequent longitudinal assessment of liver health, it has associated drawbacks related to additional patient time in the scanner, patient discomfort, and added costs (e.g., infrastructure and patient charge-related).

Increasing literature has shown that modern machine learning techniques have shifted from focusing primarily on computer-aided diagnosis to segmenting organs and lesions, image processing, classifying patients or lesions, and even prediction of outcomes.¹⁹⁴⁻²⁰⁰ These newer techniques may ultimately enable objective automated diagnosis and prognostication for individual patients. Previously, we developed a support vector machine classifier that is able to categorically classify liver stiffness using clinical and non-stiffness MRI radiomic features in pediatric and young adult patients with known and suspected chronic liver disease. Such an algorithm could theoretically decrease the use of MRE in patients with predicted normal liver stiffness, thereby decreasing imaging time and healthcare costs. However, in this prior study, we extracted handcrafted radiomic features (e.g., histogram, geometric, and texture metrics of MR images) from manually segmented livers from axial T2-weighted fat-suppressed MR images. This handcrafted image feature extraction process is time consuming and might fail to recognize certain important non-hepatic image features indicative of liver stiffening (e.g., splenomegaly, varices, ascites), potentially leading to a sub-optimal performance. Meanwhile, deep learning has demonstrated state-of-the-art performance for medical imaging analysis,^{199,201,202} providing an opportunity to utilize the original axial T2-weighted fat-suppressed MR images of the liver and surrounding structures directly, without the need for manual segmentation or radiomic feature extraction.

### SUMMARY

An object is to provide clinically-effective computer-aided diagnosis techniques to help interpret liver MRI, providing a quantitative assessment of CLD. More specifically, an object is to apply DL methods to non-elastographic MRI, MRE, and clinical data to accurately detect and quantify LF, using biopsy-derived histologic data as the reference standard. In an embodiment, we will leverage a multi-center database of several thousand pediatric and adult liver MRI examinations from four institutions that include MRE, with ≥15% having correlative biopsy data. We will validate and test the models using independent, multi-vendor datasets and will utilize DL to identify those imaging and clinical features that are most highly predictive of LF.

It is an object to provide an automated DL framework to extract radiomic and deep features from multiparametric MRI. Radiomic features (mathematical constructs capturing the spatial appearance and spectral properties tissues through imaging descriptors of gray-scale signal intensity distribution, shape and morphology, and inter-voxel signal intensity pattern/texture) and deep features (complex abstractions of patterns learned from input images through multiple non-linear transformations estimated by data driven DL training procedures) allow detection of liver and spleen structural abnormalities/tissue aberrations. In an embodiment, a special type of U-shaped convolutional neural network (CNN) is provided with both Short and Long Residual connections (SLRes-U-Net) to simultaneously take multiparametric MRI as inputs and jointly segment liver and spleen. Using the segmentations, we will 1) run an established PyRadiomics pipeline to extract MRI radiomic features; and 2) implement a pre-trained very deep CNN (e.g., GoogLeNet, ResNet) to extract MRI deep features.

It is an object to provide an “ensemble” DL model (LFNet) to predict biopsy-derived LF stage and LF percentage using the integration of multiparametric MRI radiomic and deep features, MRE, and routinely-available clinical data. An embodiment will train a series of prognostic models by applying different feature sets and classification algorithms. The LFNet will then be developed by aggregating all the models we train. This “wisdom of crowds” approach combines multiple models to fill in each other's weaknesses, therefore rendering better performance over each individual one. Clinical data will be related to three domains: i) demographic/anthropo-morphic data (e.g., age, sex, BMI); ii) diagnoses (e.g., diabetes, viral hepatitis), and iii) laboratory testing (e.g., ALT, AST, bilirubin). We will employ saliency map and feature ranking approaches to decode the LFNet model to identify the most discriminative imaging features and clinical risk factors of LF.

It is an object to provide a DL model (LSNet) to quantify MRE-derived LS using multiparametric MRI radiomic and deep features as well as routinely-available clinical data. In an embodiment a multi-channel deep neural network model is provided, simultaneously using multiparametric radiomic and deep features±clinical data as inputs, to predict the MRE-derived LS.

Embodiments of the current disclosure will significantly impact public health because it will allow physicians and researchers to more accurately evaluate millions of Americans with or at risk for CLD and LF as well as permit more frequent noninvasive, patient-centric assessment, thereby potentially improving patient outcomes and lowering healthcare costs. Developed embodiments also will be broadly applicable to the prediction of other important liver-related clinical outcomes, including impending complications such as portal hypertension, time to liver transplant/transplant listing, and mortality risk, among others.

An aspect of the current disclosure provides a method for performing a medical diagnosis of liver diseases comprising the steps of: receiving MRI data and clinical data concerning a patient's liver; diagnosing aspects of liver disease by applying a machine learning engine to the MRI data and clinical data, wherein the machine learning engine uses biopsy-derived histologic data as a reference standard; and communicating detected and quantified liver disease aspect information to a user. In a more detailed embodiment, the machine learning engine extracts and integrates radiomic features and deep features from the MRI data in the diagnosing step. In a further detailed embodiment, the MRI data represents segmented portions of the liver and spleen. In a further detailed embodiment, the diagnosing step utilizes a convolutional neural network provided with both Short and Long Residual connections (SLRes-U-Net) to simultaneously take MRI as inputs and jointly segment the liver and spleen. Alternatively, or in addition, the radiomic features comprise constructs capturing spatial appearance and spectral properties of tissues through imaging descriptors of grey-scale signal intensity distribution, shape morphology, and inter-voxel signal intensity pattern. Alternatively, or in addition, the deep features comprise complex abstractions of patterns learned from input images through multiple non-linear transformations estimated by data driven deep learning training.

In another detailed embodiment, the receiving step also receives MRE data; and the diagnosing step diagnoses liver disease by applying a machine learning engine to the MRI data, MRE data and clinical data. In a further detailed embodiment, the diagnosing step predicts biopsy-derived liver fibrosis stage and liver fibrosis percentage. Alternatively, or in addition, the clinical data comprises demographic data, diagnosis data and laboratory testing data.

In another detailed embodiment, the diagnosis step predicts MRE-derived shear LS utilizing a DL regression model on at least the MRI data. In another detailed embodiment, the method further comprises a step of training the machine learning engine using transfer learning. In another detailed embodiment, method further comprises a step of training the machine learning engine using ensemble learning.

In another detailed embodiment, the machine learning engine of the diagnosing step segments liver and spleen using a convolutional neural network provided with both short and long residual connections to extract radiomic and deep features from the MRI data. In a further detailed embodiment, the diagnosing step further implements data augmentation as part of the liver and spleen segmenting process.

In another aspect, a system is provided for performing a medical diagnosis of liver disease, where the system includes: one or more sources of MRI data and clinical data concerning a patient's liver; a machine learning engine configured to receive the MRI data and clinical data and diagnosing aspects of liver disease by applying one or more machine learning models to the MRI data and clinical data; and a computerized output communicating detected and quantified liver disease aspect information from the machine learning engine to a user. In a detailed embodiment, the machine learning engine extracts and integrates radiomic features and deep features from the MRI data in the diagnosing step. In a further detailed embodiment, the MRI data represents segmented portions of the liver and spleen. In a further detailed embodiment, the machine learning engine comprises a convolutional neural network provided with both short and long residual connections to simultaneously take MRI as inputs and jointly segment the liver.

In an embodiment, the one or more sources further include MRE data; and the machine learning engine is configured to diagnoses liver disease by applying the one or more machine learning models to the MRI data, MRE data and clinical data. In a further detailed embodiment the machine learning engine is configured to predict biopsy-derived liver fibrosis stage and liver fibrosis percentage. In a further detailed embodiment, the clinical data comprises demographic data, diagnosis data and laboratory testing data.

In an embodiment, the machine learning engine comprises a convolutional neural network provided with both short and long residual connections to extract radiomic and deep features from the MRI data to segment the liver and spleen. In a further detailed embodiment, the machine learning engine implements data augmentation as part of the liver and spleen segmenting process. Alternatively, or in addition, the machine learning engine includes a u-shaped convolutional neural network provided with both short and long residual connections to simultaneously take MRI data as input to jointly segment the liver and spleen. In a more detailed embodiment, the convolutional neural network includes a symmetric architecture, having an encoder that extracts spatial features from the MRI data, and a decoder that constructs a segmentation map. In a further detailed embodiment, the convolutional neural network includes a 3-dimensional convolutional block and a 3-dimensional residual block. In a further detailed embodiment, the convolutional 3-dimensional convolutional block includes a 3-dimensional convolution layer, an instance normalization layer and a leaky rectified linear unit later. Alternatively, or in addition, the 3-dimensional residual block includes an additional short residual connection, linking input with output feature maps of the residual block and performing a summation operation. Alternatively, or in addition, the convolutional neural network includes an encoder that extracts spatial features from the MRI data, the encoder including a sequence of 3-dimensional convolutional blocks and a 3-dimensional residual blocks. In a further detailed embodiment, the sequence of blocks is followed by a down-sampling operation that is repeated multiple times, and after the down sampling operation at each level, the number of features channels is doubled. In a further detailed embodiment, the convolutional neural network includes a decoder that constructs a segmentation map, the decoder including a succession of 3-dimensional convolutional blocks and 3-dimensional residual blocks, which up-sample feature maps and reduce the number of feature channels by half at each successive level.

It is another aspect to provide a method for performing a medical diagnosis of liver disease, where the method includes the steps of: receiving MRI data, MRE data and clinical data concerning a patient's liver; applying a plurality of machine learning models to the MRI data, MRE data and clinical data; combining the plurality of machine learning models into an ensemble deep learning model; diagnosing aspects of liver disease based upon an output of the ensemble deep learning model; and communicating liver disease aspect information to a user. In a further detailed embodiment, the combining step includes a step of identifying, for each of the plurality of machine learning models, each model's predictive feature identification process by applying deep learning feature ranking and saliency map approaches.

In another aspect, embodiments provide a deep learning framework to accurately segment liver and spleen using a convolutional neural network with both short and long residual connections to extract their radiomic and deep features from multiparametric MRI. Embodiments will provide an “ensemble” deep learning model to quantify biopsy-derived liver fibrosis stage and percentage using the integration of multiparametric MRI radiomic and deep features, MRE data, as well as routinely-available clinical data. Embodiments will provide a deep learning model to quantify MRE-derived liver stiffness using multiparametric MRI, radiomic and deep features and routinely-available clinical data.

These and other aspects or embodiments of the current disclosure will be apparent from the following detailed description and the attached figures.

### BRIEF DESCRIPTION OF THE DRAWINGS

FIG. 1 is a high-level block diagram representation of an exemplary embodiment of a multi-model deep-learning approach for performing a medical diagnosis of the liver;

FIG. 2 is a block diagram representation of an exemplary embodiment of a system and method for performing a medical diagnosis of the liver;

FIG. 3 is a flowchart representing internal and external model validation that will work for all disclosed models, including the exemplary embodiment of FIG. 2;

FIG. 4 shows liver segmentation using an exemplary U-Net convolutional neural network model;

FIG. 5 illustrates an architecture of the exemplary 3D SLRes-U-Net for multi-organ segmentation using multiparametric MRI data;

FIG. 6 is a graph illustrating performance of an ensemble learning approach as compared to individual classifier models;

FIG. 7 is a block diagram illustrating architecture of an exemplary ensemble LFNet model for liver fibrosis prediction;

FIG. 8 is a block diagram illustrating architecture of an exemplary deep learning system/method for classifying patients into groups of liver stiffening (DeepLiverNet);

FIGS. 9A, 9B and 9C respectively provide saliency maps showing discriminative image regions ranked by deep learning prognostic models;

FIG. 10 provides architecture of the exemplary LSNet model for liver stiffness quantification;

FIG. 11 is a block diagram illustrating further detailed architecture of the exemplary deep learning system/method for classifying patients into groups of liver stiffening (DeepLiverNet) shown in FIG. 8;

FIGS. 12A and 12B respectively illustrate original liver images (12A) and three randomly synthesized liver images (12B) from three different subjects using the rotation and shift-based data augmentation algorithm; and

FIG. 13 provides internal and external validation experiments flow chart for DeepLiverNet.

### DETAILED DESCRIPTION

### A. Significance

A.1. Impact on public health. Chronic liver disease (CLD) is a common cause of morbidity and mortality in the United States (U.S.) and throughout the world. According to the U.S. Centers for Disease Control and Prevention, CLD had an age-adjusted death rate of 10.9/100,000 total population in 2017, increased from 8.9/100,000 total population in 2005.¹ In certain states, such as New Mexico, the rate is as high as 26.8/100,000 total population. Based on a study by Younossi et al.² using the National Health and Nutrition Examination Survey data, the prevalence of non-alcoholic fatty liver disease (NAFLD) has increased from 20% (1988-1994) to 32% (2013-2016) and is widely accepted as the most prevalent form of CLD worldwide.³ The prevalence of NAFLD is also rapidly increasing in the pediatric population, with a global prevalence approaching 10% and a prevalence of 34.2% in obese children.⁴ In the U.S. where over 64 million people are projected to have NAFLD, the estimated annual direct medical costs have been estimated to be $103 billion.⁵ When considering other causes of pediatric and adult CLD, the costs of these diseases is $100s billion worldwide. Ongoing liver inflammation and hepatocyte injury leads to aberrant healing and variable liver fibrosis (LF) in most CLD patients. This process is unpredictable between individuals varying in rate of progression and severity. Over time, many CLD patients go on to develop cirrhosis, portal hypertension (commonly manifested as portosystemic varices, splenomegaly, and ascites), and end-stage liver disease, with considerable associated morbidity and mortality and nearly 10,000 individuals receiving a liver transplant each year in the U.S. (˜500-1000 are children).⁶

A.2. Rigor of prior research. Data from one NIH-funded R21 project (Pl: He) along with two institution-funded projects (Pl: Dillman, He) have generated unique insights that have informed the development of this application.⁷⁻¹⁸ Six points are significant:

1) Biopsy is limited for evaluation of CLD: Percutaneous (and less often transjugular or intraoperative) liver biopsy with histopathologic assessment remains the reference standard for detecting and quantifying (staging) liver fibrosis. However, liver biopsy has noteworthy limitations, including sampling error (only a very tiny volume of tissue can be sampled and many CLD do not affect the liver uniformly, and, therefore, 1) severity of LF may be under- or overestimated, and 2) significant changes over time can be difficult to conclusively establish), imperfect inter-pathologist agreement, risk of morbidity and uncommonly mortality, and relatively high cost.¹⁹ Biopsy also can be uncomfortable and even painful, limiting its use in longitudinal monitoring of CLD severity and LF progression. Thus, there is a highly compelling need to develop noninvasive CLD biomarkers. We, along with other researchers, have independently described noninvasive biomarkers for evaluating LF, including serum biomarkers from laboratory tests (e.g., aspartate aminotransferase (AST)-platelet ratio index (APRI) and FIBROSIS-4 score)²⁰⁻²² and elastographic liver stiffness (LS) measurements from medical imaging.⁸

2) LS as measured using MR Elastography (MRE) is an emerging biomarker of LF, but has important limitations: Although MRE obviates the need for liver biopsy in some patients and allows more frequent longitudinal assessment of liver health, it has drawbacks related to additional patient time in the scanner, mild patient discomfort, and added costs (e.g., infrastructure [˜$100,000-250,000 per MRI scanner to setup] and patient charge-related). MRE also has variable diagnostic performance based on the literature, and LS is a confounded biomarker, impacted by fibrosis, venous congestion, inflammation, and fat.^{23, 24} We have successfully demonstrated that machine learning (ML)/deep learning (DL) techniques can classify the severity of LS as determined by MRE using non-elastographic MR imaging data (e.g., T2-weighted images).^{14, 18}

3) Radiomics has tremendous potential to quantify subtle disease: Radiomics is the high throughput extraction of quantitative imaging features followed by advanced image processing and analysis techniques that can decode image-based aberrations due to histologic tissue changes for potentially improved detection of disease and decision support.^{14, 25-28} Extracting radiomic features involves a complex, two-step process, including segmentation of regions of interest and feature quantification. Automatic segmentation is challenging because of reproducibility. We have prior experience developing DL segmentation methods,¹¹ including our deep U-Net convolutional neural network (CNN) for liver segmentation which has achieved a mean Dice similarity coefficient (DSC) of >0.90. In addition, validated software, such as PyRadiomics,²⁹ has been developed to objectively quantify radiomic features.

4) Deep features compliment radiomic features for disease diagnosis and prognosis: With increasing computational efficiency, DL techniques are poised to facilitate major breakthroughs in the medical field, aiding in diagnosis, disease classification, outcome prediction, and treatment decision making. DL provides a class of artificial neural networks (ANN)³⁰ to model complex abstractions of patterns, i.e., “deep features” through multiple non-linear transformations determined by data-driven training procedures. Our group has demonstrated that deep features have predictive capabilities for disease diagnosis and prognosis.^{12, 15, 16}

5) Transfer learning improves DL performance: We have shown considerable success in developing DL-based prognostic/diagnostic and segmentation models using MRI data for a variety of medical applications.^{12, 15, 16} To overcome the fundamental challenge of insufficient training data in DL,³¹⁻³⁶ we recently demonstrated that transfer learning, the act of repurposing a previously trained model for a different task, is an effective strategy to enhance DL model training with limited data for prediction of cognitive deficits, autism spectrum disorder, and stroke recovery.¹⁵⁻¹⁷

6) “Ensemble” learning enables feature integration: In practice, many ML classification models are available for prediction of medical outcomes, although most lack sufficient diagnostic accuracy to be relied upon in the clinical setting. Studies have demonstrated that aggregation of multiple models through an ensemble model can produce superior performance compared with each individual model.³⁷⁻⁴² Ensemble models also allow the integration of multiple unique feature-types, such as multiparametric MRI and clinical data.

Together, these supporting data and publications provide a basis for the disclosed embodiments of applying DL methods using MRI, MRE data, and readily-available clinical data for accurate detection and quantification of CLD severity and LF in children and adults.

A.3. Impact on personalized medicine and clinical trials. Accurate diagnosis of CLD remains challenging, with the severity of fibrosis directly related to key clinical outcomes. Accurate and reproducible noninvasive prediction of LF will be a milestone, closing a gap needed for improved clinical care of millions of Americans suffering from CLD. The disclosed techniques will enable the development of clinically relevant disease and risk prediction models that will allow more frequent monitoring of CLD to assess for treatment response/disease progression, potentially improving overall liver-related outcomes and lowering healthcare costs. Costs could be lowered in a variety of ways, including less frequent CLD-related complications, fewer liver transplantation procedures, and decreased need for invasive liver biopsies and elastographic imaging. Ultimately, disclosed embodiments will enhance our abilities to stage CLD in a quantitative, noninvasive, patient-friendly manner as well as to provide more patient-centric, precision medicine. Furthermore, disclosed models could be used as endpoints in therapeutic clinical trials, potentially decreasing the need for liver biopsies and reducing variability in CLD severity measurements which could lower the number of patients required for a given study.

B. Innovation. This disclosure will provide a very desirable framework to the community, allowing clinicians to rapidly and noninvasively detect and measure the severity of CLD and LF. To achieve this, embodiments will combine computer science, MR imaging, diagnostic radiology, hepatology, biomedical engineering, and biostatistics. Embodiments disclosed herein are groundbreaking in multiple ways:

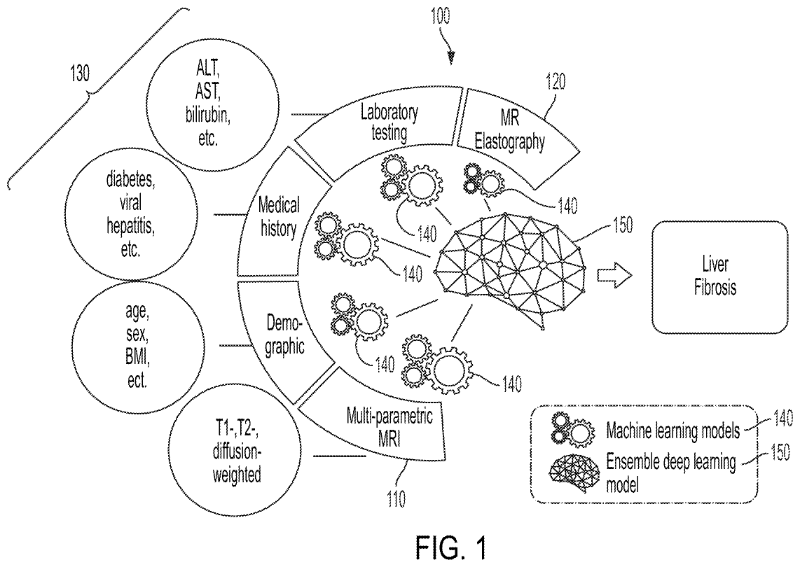

B.1. Ensemble multi-model DL approach. Referring to FIG. 1, the disclosed ensemble DL model 100 for quantifying CLD severity and LF will use multiparametric MRI 110, MRE 120, and routinely-available clinical data 130. Specifically, each of these data input types will be used to create multiple unique ML models 140 (e.g., logistic regression,⁴³ random forest,⁴⁴ support vector machine (SVM),⁴⁵ ANN) that will then be combined into a single ensemble DL model 150.

This “wisdom of crowds” approach combines multiple models to fill in each other's weaknesses, therefore rendering better performance over each individual one.³⁷ This approach is novel to the problem at hand and may provide the highest possible accuracy for noninvasively detecting and quantitatively estimating severity of LF by integrating all available data in a rigorous manner.

B.2. Integration of MRI radiomic and deep features. Conventional MRI enables noninvasive detection and characterization of liver pathology. It has become an increasingly important clinical imaging modality for the investigation of patients with CLD.⁴⁶⁻⁴⁹ Radiomics, an emerging translational field in radiology, is defined as the high throughput extraction of quantitative imaging features to build a signature with the aid of advanced image processing and analysis techniques for improved characterization of tissue pathology and diagnosis.^{25, 50} MRI radiomic features, which are generally unable to be quantified by the human eye and brain, provide descriptors of signal intensity distribution, organ (e.g., liver and spleen) morphology/shape, volumetry, and inter-voxel patterns and texture. These objectively quantified and interpretable MRI radiomic features^{29, 51} change with alterations in tissue histology and morphology, thereby enabling automatic evaluation of disease severity and potentially informing therapeutic decisions. On the other hand, DL techniques, based on ANN, provide a sophisticated and rigorous means to model complex abstractions of patterns through multiple non-linear transformations estimated by data-driven training procedures. Deep features are pathologically meaningful features extracted by DL to reveal discriminative information from high dimensional medical imaging data. Although deep features generally lack interpretability (unlike radiomic features), such latent features can complement and possibly outperform radiomic features in the outcome prediction.^{52, 53}

Accurate analysis of the integration of quantitative MRI radiomic and deep features affords unique opportunities to gain a better understanding of how organ tissue characteristics and their pathology information are segregated and integrated. Exemplary models will extract radiomic and deep features from both the liver and spleen to quantify CLD severity and LF. The addition of splenic features is based on existing literature showing that splenic size (length/volume) and stiffness changes occur with increasing CLD severity and onset of portal hypertension.⁹ Furthermore, recent work from the current inventors shows that changes in splenic T1 relaxation time are associated with CLD severity.⁵⁴

In summary, capturing meaningful information contained in MRI data, together with developing noninvasive diagnostic tools, highlight the discipline of integration of radiomic and deep features as well as utilizing DL for MRI studies. Application of DL to liver MRI as disclosed herein may create entirely new insights into the accurate diagnosis of CLD and quantification of LF, and enhance the move towards precision medicine.

B.3. One of the largest diverse liver MRI-pathology datasets. Application of an embodiment will have likely have created one of the largest (if not the largest) multi-center (Cincinnati Children's Hospital Medical Center [CCHMC], University of Wisconsin [UW], University of Michigan [UM], and New York University [NYU]) liver MRI-pathology datasets that is composed of both anatomic and MRE images. This dataset will include several thousand clinical liver MRI exams from all three major manufacturers (GE Healthcare, Philips Healthcare, and Siemens Healthcare) as well as acquisitions obtained on both 1.5T and 3T clinical MR systems. This dataset will include large numbers of scans and correlative biopsy tissue from pediatric and adult populations as well as from patients with a variety of causes of CLD (e.g., NAFLD/non-alcoholic steatohepatitis [NASH], viral hepatitis, autoimmune liver diseases, and biliary atresia).

B.4. Transfer learning prevents DL model overfitting. DL has unique ability to fit a variety of complex datasets with great freedom, thanks to the huge number of model parameters (thousands to millions). This unique ability allows DL to outperform “traditional” ML when solving complex problems with “big data”. However, this advantage may also represent a potential weakness. Lack of control over the model training process may lead to overfitting when the DL model is so closely fitted to the training set that it fails to generalize and make accurate predictions for new data.⁵⁵⁻⁵⁸ Transfer learning^{59, 60} is an important key to solve the fundamental problem of overfitting in DL.³¹⁻³⁶ Transfer learning will repurpose models developed for other tasks to ultimately improve the performance and generalizability of new models as well as decrease the amount of data needed for model training. Transfer learning-augmented DL models may show improved model fidelity and, thus, impact medical diagnosis in the same way as DL has revolutionized other fields (e.g., image recognition^{58, 61} and speech recognition^{62, 63}).

B.5. Illuminating the “black box” nature of DL methods. Despite DL's many practical successes, there is still skepticism regarding its clinical adoption emanating from its ‘black box’ nature. The inability to understand a model (with millions of model parameters) can lead to mistrust and limit confidence in the method, and, thus, is it may present a barrier to the clinical translation of such techniques.⁶⁴⁻⁶⁶ There has been increasing effort in making DL methods more transparent. In theory, the DL model compresses input data as if by squeezing the information through a bottleneck, retaining only the features most relevant to the learning task.^{67,68} The compression process is pronounced at the DL model's deeper layers where information relevant to the output labels is preserved at the expense of gradually “forgetting” input information. In practice, methods have been proposed to decompose model decisions in terms of inputs.⁶⁹

In the current disclosure, we unravel and illuminate the DL models' predictive feature identification process by applying DL feature ranking and saliency map approaches.⁷⁰⁻⁷⁶ In addition, expert-evaluators further validate the DL-identified discriminative features. Such an approach may generate greater trust in the models from clinicians for eventual translation to the bedside. This procedure also may enable users to discern a “stronger” model from a “weaker” one, even when both models make identical predictions. The explanatory approach to model understanding in conjunction with model validation and testing using independent external datasets may establish users' trust in the predictions made by DL models.^{67, 71} This may be important for accelerating clinical translation of DL personalized medicine.

B.6. Model generalizability. In addition to creating agnostic, generalizable models that allow input of pediatric and adult data from any given form of CLD to predict/quantify LS and LF, embodiments of the current disclosure may create models that are unique to specific CLD subpopulations (e.g., adult or pediatric NAFLD, adult viral hepatitis). Furthermore, exemplary DL models may be used to predict other important clinical outcomes in CLD (e.g., onset of impending complications, such as portal hypertension, time to transplant/transplant listing, and mortality) and characterize a variety of other non-liver chronic medical conditions.

### C. Approach

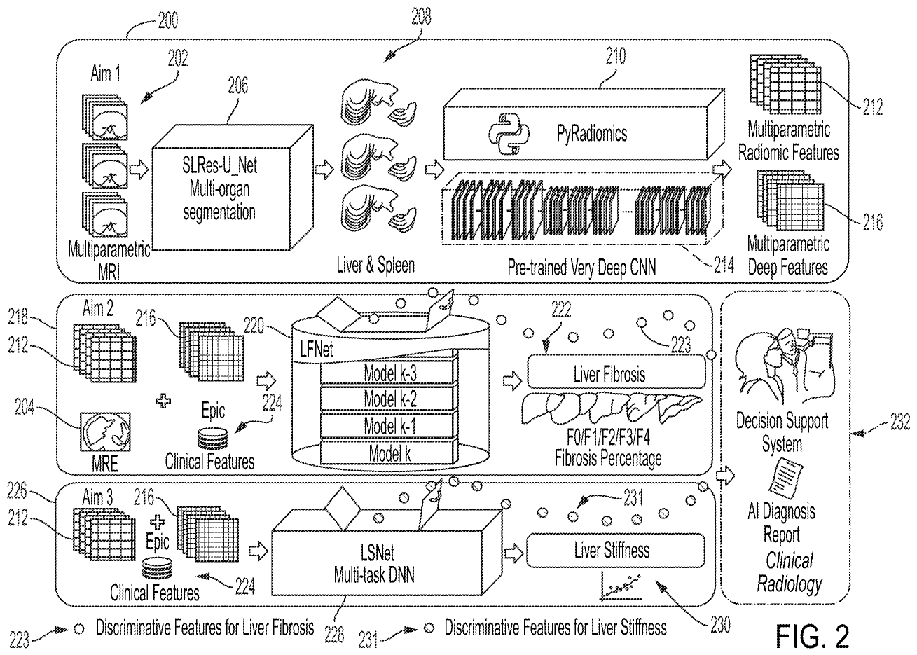

C.1. Overview. A conceptual overview of embodiments of the current disclosure incorporating three aims is shown in FIG. 2. For patients with chronic liver diseases (CLD), embodiments will utilize multiparametric MRI 202, MR elastography (MRE) 204, and correlative histologic data. In Aim 1 200, embodiments will provide a deep learning framework to accurately segment liver and spleen using SLRes-U-Net 206 to extract their radiomic and deep features from multiparametric MRI 202. The SLRes-U-Net simultaneously takes multiparametric MRI 202 (e.g., T1-, T2-, and diffusion-weighted images) as inputs and jointly segments liver and spleen 208. Based on the segmentations, such embodiments will run a well-established PyRadiomics pipeline 210 to extract radiomic features 212 as well as implement a pre-trained very deep convolutional neural network 214 (CNN, e.g., GoogLeNet, ResNet) to extract deep features 216. In Aim 2 218, embodiments will provide an “ensemble” deep learning model (LFNet) 220 to quantify biopsy-derived liver fibrosis stage and percentage 222 using the integration of multiparametric MRI 202 radiomic and deep features 212, 216, MRE data 204, as well as routinely-available clinical data 224. Such outputs 222 may be communicated to the user via computer display, electronic messaging, print-out, or any other known mechanism for communication. In Aim 3 226, embodiments will provide a deep learning model (LSNet) 228 to quantify MRE-derived liver stiffness 230 using multiparametric MRI 202, radiomic and deep features 212, 216 and routinely-available clinical data 224. By decoding each model, embodiments will identify, validate, and disseminate a series of the most discriminative imaging and clinical features to the community. Outputs 232 from Aim 2 and/or Aim 3 may include a decision support system and/or an AI Diagnosis Report for clinical radiology. Such outputs 232 may be communicated to the user via computer display, electronic messaging, print-out, or any other known mechanism for communication. The techniques will enhance our abilities to assess CLD in a quantitative, noninvasive, patient-friendly manner as well as to provide more patient-centric, precision medicine.

C.2. Scientific rigor. Development of the disclosed embodiments followed the guidance for radiology research on artificial intelligence provided by the journal Radiology Editorial Board⁷⁷ to achieve robust, unbiased, and reproducible results: 1) Exemplary DL models will be trained, validated, and tested on large independent datasets without overlap; 2) To reduce the possibility of model overfitting, exemplary embodiments will utilize previously described transfer learning¹⁵ and data augmentation techniques to increase training datasets;^{61, 78, 79} 3) To assess the generalizability of current approach, exemplary embodiments will test the DL models using independent external datasets pulled two years later;⁷⁷ 4) To ensure model robustness, exemplary embodiments will use multivendor (including 1.5T and 3T field strengths) and multisite datasets for the model development; 5) All liver tissue biopsy specimens will be centrally reviewed and scored by expert study hepatopathologists in order to ensure the best possible reference standard; 6) Multisite and multivendor datasets will be objectively harmonized and preprocessed using in-house established pipelines,⁸⁰ 7) Relevant clinical features will be extracted from the electronic medical records of each subject using automated methods. All participating institutions use the same electronic medical record system (Epic Systems Corporation; Verona, Wis.); 8) MRI radiomic and deep features will be objectively quantified with high reproducibility using well-established, automated pipeline and pre-trained DL models;^{29, 81} 9) Prognostic models will be fully automated without human intervention; and 10) To increase interpretability, exemplary embodiments will use accepted methods to identify the most discriminative clinical, radiomic, and deep features of LF and LS.^{14, 70-76} Expert-evaluators will further validate the DL-identified discriminative features.

C.3. Consideration of sex and other clinical variables. While CLD affects both men and women, it is a complex group of disorders that may have sex-related differences. It is noteworthy that sex is a potential biomarker of CLD, with a higher age-adjusted death rate in male patients (14.3/100,000 population) compared to female patients (7.5/100,000 population). For this reason, sex is a biological variable that will be considered in some embodiments to further enhance scientific rigor. Embodiments may calculate the diagnostic accuracy of clinical variables for predicting LF and LS as well as will integrate clinical features with MRI radiomic and deep features to further boost the DL model performance and enhance scientific rigor. Clinical features may be related to three overarching domains: demographic and anthropomorphic data (e.g., sex, age, body mass index), medical history and specific clinical diagnoses (e.g., diabetic status, specific chronic liver diseases, such as viral hepatitis), and laboratory testing (e.g., alanine aminotransferase level, aspartate aminotransferase level, bilirubin level, albumin level, platelet count, APRI score, and FIBROSIS-4 score).

C.4. Study Design Elements Common to All 3 Aims

C.4.1 Subjects and MRI acquisition. Embodiments create and harmonize a very large, multi-vendor (GE Healthcare, Philips Healthcare, and Siemens Healthcare), multi-field strength (1.5T and 3T), multi-center (CCHMC, UW, UM, and NYU) liver MRI dataset that is composed of both anatomic and MRE (including both gradient recalled echo and spin-echo echo-planar imaging data) images. The dataset includes ˜1,500 pediatric (0-18 years of age) and 6,000 adult MRI examinations. Liver and spleen segmentations on 1500 examinations will serve as ground-truth for segmentation model development. Examinations are from patients with a variety of CLD, with known or suspected fatty liver disease (NAFLD/NASH) and viral hepatitis being most common. All MRI examinations include clinical noncontrast T1-weighted (gradient recalled echo), T2-weighted (single-shot fast spin-echo, multi-shot fast spin-echo) as well as chemical shift-encoded multi-echo Dixon (e.g., IDEAL IQ, mDixon Quant) imaging providing proton density fat fraction. The majority of exams also include diffusion-weighted imaging (DWI) data, with the upper b-value ranging from 600-800 s/mm². These imaging data will be used for all three aims, including both LF and LS quantification.

C.4.2. Histologic LF assessment. Based on institutional searches of radiology and pathology records during the preparation of this application, it is anticipated that ˜15% of subjects (˜1,125 subjects) with relevant MRI data will have contemporaneous correlative liver biopsy tissue available for assessment. Available tissue specimens in the form of existing stained slides (including Masson trichrome or Sirius red stained), recut unstained slides, and/or paraffin blocks will be obtained. All recut unstained slides will undergo staining as a batch using a fibrosis-specific stain (e.g., Masson's trichrome). At least two slides from each subject will be reviewed separately and scored for the presence and amount of fibrosis by two study expert hepatopathologists using a validated semi-quantitative staging system (e.g., METAVIR).⁸² Slides also will undergo digital scanning and the fibrosis percentage (0-100%) on each slide will be quantified as measured by the collagen proportionate area⁸³ using an existing computer-based algorithm⁸⁴; two slides will be scanned per subject with the fibrosis percentage averaged.

C.4.3. MRI data harmonization. Utilizing MRI datasets from multiple clinical sites and MRI scanners will improve statistical power and the generalizability of results. However, multi-site MRI examinations have reported nonbiological variability in image features due to the technical variation across different scanners, magnetic field strengths, and acquisition protocols.⁸⁵ Thus, embodiments may apply a harmonization technique called ComBat⁸⁰ to remove such undesirable variabilities. ComBat was originally designed to correct so-called “batch effects” in genomic studies that arise due to processing high-throughput genomic data in different laboratories with different equipment at different times. It has recently been shown that this harmonization method is a reliable and powerful technique that can be widely applied to different imaging modalities and radiomics measurements and is successful in eliminating site effects in multi-site structural MRI quantitative data.⁸⁶⁻⁸⁸

C.4.4. Supervised transfer learning implementation. Embodiments may utilize models developed for other tasks to ultimately improve the performance and generalize-ability of the exemplary models as well as decrease the amount of data needed for model training. More specifically, a pre-trained very deep CNN model may be implemented without its original classifier for deep feature extraction (Aim 1 200); a new classifier that fits the purpose (LF and LS quantification) may be added, freeze the pre-trained model, and then only train the new classifier (Aim 2 218 and Aim 3 226). The candidate pre-trained deep CNN models may include the winning models from the annual (2010-2017) ImageNet Large Scale Visual Recognition Challenge (ILSVRC)⁸¹ competition. This competition was designed to foster the development of computer vision algorithms using ˜1.2 million natural images from the ImageNet database.⁸⁹ The ImageNet pre-trained models to implement and compare may include VGG,⁹⁰ ResNet,⁹¹ ResNetV2,⁹² ResNetXt,⁹³ Inception,⁹⁴ InceptionResNet,⁹⁵ DenseNet,⁹⁶ and NASNet.⁹⁷

C.4.5. DL model architecture optimization. DL modal optimization involves the determination of a set of hyperparameters (the numbers of hidden layers and neurons at each layer).^{58, 98} The model performance depends upon these architectural attributes. A model with few layers and neurons can lead to underfitting (poor performance on the training data and poor generalization to other data), while too many layers and neurons can lead to overfitting (good performance on the training data and poor generalization to other data). As the combinations of the hyperparameters can be huge and each corresponds to a network training, brute force search is prohibitive and nonlinear optimization is preferred.^{97, 99-102} Following the approach proposed by IBM research,¹⁰³ embodiments will adopt a global optimization with continuous relaxation approach for the SLRes-U-Net model 206 optimization (Aim 1). In addition, embodiments will implement multiple different automated optimization algorithms that are specifically designed for DL and adopt the best one for the proposed LFNet 220 (Aim 2) and LSNet 228 (Aim 3) models. The candidate algorithms may include: reinforcement learning neural architecture searching,¹⁰⁰ neural architecture optimization algorithm,¹⁰² and differentiable architecture search.¹⁰⁴

C.4.6. DL model training. For supervised model training, embodiments may utilize a DSC as loss function for segmentation (Aim 1), and the sum of a cross-entropy and mean square error (MSE) as a multi-task loss function for joint classification and regression (Aims 2 and 3). For unsupervised training in transfer learning, embodiments may apply a Kullback-Leibler divergence regularized MSE as loss function. A mini-batch gradient descent algorithm may be chosen to minimize the loss function so as to optimize the model weights. This mini-batch variation of training algorithm divides the training data into small batches and updates the model weights using only data from every batch, enabling a faster, but more stable convergence for training. The batch size will be calculated during the optimization. Candidate gradient descent algorithms may include stochastic gradient descent,¹⁰⁵ Adam algorithm,¹⁰⁶ RMSprop,¹⁰⁷ and Adagrad.¹⁰⁸ The weights of convolutional and fully-connected layers may be randomly initialized using Glorot uniform distribution.¹⁰⁹ The number of training epochs may be set with an early stop mechanism that will cease the optimization process if several consecutive epochs return the same loss errors based on validation data. The initial learning rate will be set based on the performance after testing several empirical values (e.g., 0.001, 0.01. 0.05, 0.1, 0.5).

C.4.7. DL model illumination. DL feature ranking and saliency map approaches⁷⁰⁻⁷⁶ may be applied to unravel and illuminate the DL models' predictive feature identification process. Heat maps visualizing the importance of each input feature utilized for prediction may be shown. This could help to further optimize the DL model and ensure it is “paying attention” to the correct discriminative features. In addition, experienced radiologists and/or hepatologists may be used to evaluate the DL-identified features to lend insight into whether significant predictions have reasonable explanations, and vice versa, to expose novel discoveries.

C.4.8. Data balancing and augmentation. Imbalanced datasets (relatively small number of patients having severe fibrosis) can negatively impact the model's learning ability.^{78, 110-113} In such cases, the models are prone to become majority class classifiers, i.e. they fail to learn the concepts of the minority class. As such, embodiments may employ modified synthetic minority over-sampling¹¹³ and adaptive synthetic sampling approach⁷⁸ to overcome this challenge. By synthetically generating more samples of the minority class, the classifiers are able to broaden their decision regions for a given minority class. In addition, for DL segmentation, patch-based data augmentation may be implemented.⁷⁹ Specifically, 3D image volumes may be parcellated (including for 2D acquisitions) into a large number of overlapping/non-overlapping patches. This may not only increase the training samples, but also decrease the dimension of input data. Rotation and shift-based data augmentation strategy may also be applied.^{61, 79, 114} The augmentation may be applied on-the-fly on the patch-level using the ImageDataGenerator function implemented in Keras. The synthesized samples may be used for model training and excluded from performance testing.

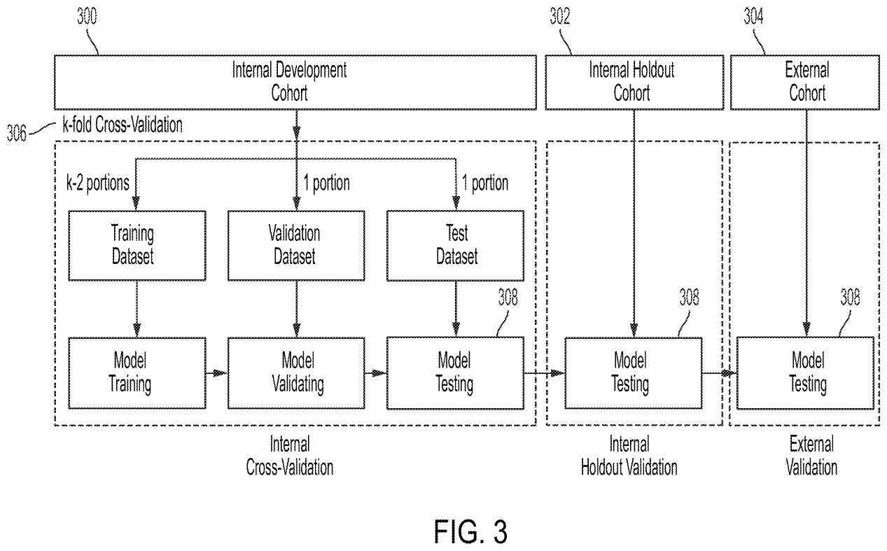

C.4.9. Model validation and assessment. As shown in FIG. 3, exemplary DL models may be trained, validated and tested using three independent datasets without overlap. Embodiments may initially pull 7,500 MRI examinations (multivendor and multisite) at year 1 and may use 80% of these data as internal development cohort 300 and reserve 20% of them to be a separate internal holdout cohort 302. At year 3, another ˜3,000 MRI examinations (multivendor and multisite) may be pulled and used as independent external cohort 304. K-fold cross-validation 306 on internal development cohort 300 may be conducted. The model may then be tested (step 308) on both internal holdout cohort 302 and external cohort 304.⁷⁷ To evaluate the performance of 1) Liver and spleen segmentation vs. manual segmentation (Aim 1 200), DSC may be reported; 2) Quantification of LF staging vs. pathologist fibrosis staging (Aim 2 218), accuracy, sensitivity, specificity, and area under the receiver operating characteristic curve (AUROC) (e.g., F0 vs. F1-4; F0-1 vs. F2-4; F0-2 vs. F3-4; etc.) with their corresponding 95% confidence interval (CI) may be calculated; 3) Quantification of LF percentage vs. computer-based assessment (Aim 2 218) and quantification of continuous LS vs. MRE-derived LS (Aim 3 226), mean absolute error, intra-class correlation coefficient, and Bland-Altman mean bias and 95% limits of agreement may be reported.

C.4.10. Power analysis and statistical analysis plan. From a previous studyl¹¹⁵, the accuracy and AUROC of predicting fibrosis stage F0-1 vs. F2-4 (F0-2 vs. F3-4 and F0-3 vs. F4) were 75% (77% and 80%) and 0.85 (0.84 and 0.84) when using a contrast-enhanced MRI sequence. It is believed that the current ensemble LFNet model, which integrates data from multiple MRI sequences, MRE, and clinical data, will improve the accuracy and AUROC to 90% and 0.95, respectively. Based on a Chi-square test with a 2-sided alpha error of 0.05, an internal holdout validation sample size of 225 patients will provide over 95% power to detect the specified difference between the proposed LFNet model and the existing method. The sample size estimate is also inflated to account for an expected 5% rate for corrupt quantitative MRI data (e.g., due to artifacts or missing sequences). Since 20% of the internal cohort will be used for internal holdout validation, a total sample size of 1125 patients is needed for entire internal cohort (Aim 2). With the same sample size calculated for Aim 2, we will have over 90% power to detect a 5% increase in DSC between our proposed SLRes-U-Net model (Aim 1) and current segmentation (C.5.2); and to detect a 10% increase in AUROC between our proposed LSNet model (Aim 3) and current LS quantification method (C.7.2). Details of DL models can be found in sections C.4.4-C.4.9 and each aim's study design section (C.5.3.1, C.6.3.1, and C.7.3.1). One sample chi-squared test will be used to compare the proposed methods and the existing methods. All statistical analyses will be performed using SAS version 9.4. A p-value less than 0.05 will be considered statistically significant.

C.4.11. Expected outcomes. It is expected that 1) SLRes-U-Net model using multiparametric MRI will produce more accurate liver and spleen segmentations than current models using single pulse sequence MRI data (Aim 1); 2) LFNet model will predict biopsy-derived LF stage and LF percentage with greater accuracy than MRE and/or MRI alone (Aim 2); and 3) quantified continuous LS by LSNet will be comparable to MRE-derived LS. Success in prediction for an independent testing dataset increases the clinical relevance of the proposed method. It is expected that such methods are generalizable across vendors, field strengths, sex, and age.

C.4.12. Potential pitfalls and alternative approaches. Given the strength of our preliminary data, it is expected to successfully predict histologic LF stage, LF percentage, and continuous LS (as determined by MRE) with high degrees of accuracy. If expected outcomes are not achieved despite successes to date, different from what are currently proposing to randomly sub-set the training data without replacement (k-fold CV), various versions of the training data will be generated by randomly sub-dividing the dataset with replacement (bootstrap) (Aim 2). In addition, radiomic and deep features extracted from additional sequences (e.g., T1-mapping, contrast-enhanced T1-weighted) will be incorporate (Aims 2 and 3). Lastly, instead of procuring liver and spleen radiomic and deep features from organ segmentations, an exemplary DL model may analyze whole images that have not undergone segmentation (Aims 2 and 3). If embodiments of the Aim 1 model perform poorly and/or are unable to achieve accurate segmentations of the liver and spleen for the extraction of radiomic and deep features, 1) additional sequences/MRI data sources (e.g., T1-mapping, contrast-enhanced T1-weighted) may be incorporated; and 2) different combinations of input pulse sequences may be employed to improve the segmentations. For embodiments that experience an inability to segment organs using DL, the liver/spleen for MRI exams may be manually segmented to facilitate the extraction of radiomic and deep features to be used in Aims 2 and 3.

C.5. Aim 1 (200). A DL framework to extract radiomic 212 and deep features 216 from multiparametric MRI.

C.5.1. Rationale. MRI radiomic features are mathematical constructs capturing the spatial appearance and spectral properties of the tissue/regions of interest through imaging descriptors of gray-scale signal intensity distribution, shape and morphology, volumetry, and inter-voxel signal intensity pattern and texture. These features have been correlated to tissue biology in various applications.¹¹⁶ Deep features are complex abstractions of patterns non-linearly constructed throughout the transformation estimated by data-driven training procedures in DL. Such latent features, which are invisible to the human eye, are also demonstrated to be associated with tissue architectural and morphological alterations.¹¹⁷⁻¹²⁰ Embodiments will extract radiomic 212 and deep features 216 from the liver as well as the spleen in order to quantify LF and LS. Although essential to extract radiomic and deep features, automated and accurate liver and spleen segmentation remains challenging due to high inter-subject variability in organ size, shape, signal intensity/appearance, and close proximity to other organs. Previous efforts have been made to perform automated abdominal organ segmentations on computed tomography and MRI.¹²¹⁻¹²⁸ However, most of the existing methods only produce moderate accuracies for a single image-type (e.g., CT images or T2-weighted non-fat-suppressed images).^{122, 123, 126, 129, 130} More recently, DL techniques have been shown great promise in abdominal organ segmentations, but mainly on CT scans.^{124, 127, 131} There has been a general lack of application on MRI data, especially multiparametric MRI. To improve the segmentation performance embodiments will provide a special type of Li-shaped CNN with both short and long residual connections (SLRes-U-Net), to simultaneously use multiparametric MRI data (e.g., T1-, T2-, and diffusion-weighted images of the abdomen) as inputs and jointly segment liver and spleen.

C.5.2. Preliminary Studies.

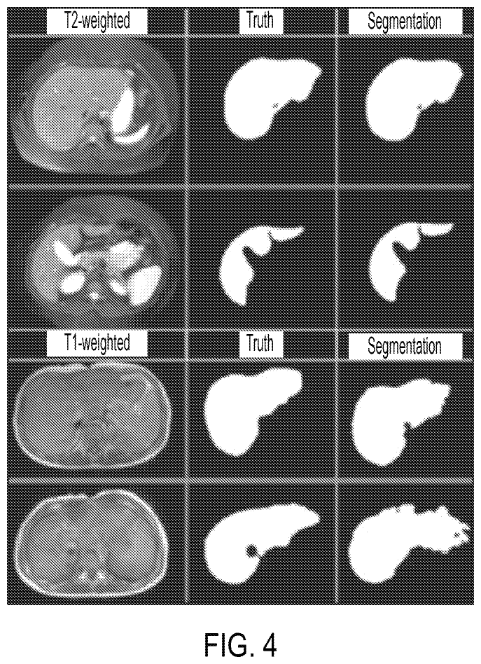

Liver segmentation using a U-Net CNN model. FIG. 4 shows liver segmentation using an exemplary U-Net convolutional neural network model. Liver segmentation at MRI can be challenging due to variability in liver morphology, motion artifacts, and low soft tissue contrast between the liver and adjacent tissue. As shown in FIG. 4, a U-Net CNN model has been developed to automatically segment liver volumes on either T2- or T1-weighted MR images. The mean age of the patients in the dataset was 14.4±6.2 years. Axial T2-weighted fat-suppressed images from 581 clinical MRI exams {˜20,000 overlapped image patches of 32×32×32 voxels) were used for training and validation. T2-weighted images from 151 patients and T1-weighted images from 15 patients (˜700 overlapped image patches of 32×32×32 voxels) were used for testing. A DSC-based loss function and the Adam optimizer were used to train the network. The proposed model resulted in a mean (standard deviation) DSC of 0.90 (0.06) on T2-weighted test set; and 0.72 (0.1) on T1-weighted test set. This ability to segment is noteworthy as training was performed using images from a pediatric population which generally has less intra-abdominal fat surrounding and separating the organs. Furthermore, the training dataset was fat-suppressed, meaning that both the liver (in the absence of moderate to severe liver disease) and surrounding fat are both relatively low in signal intensity. T1-weighted images in our multi-site dataset will be primarily gradient recalled echo as opposed to turbo (fast) spin-echo and breath-held, and thus should have less respiratory motion artifacts and a higher resultant DSC.

PyRadiomics 210 (freely available): radiomic feature quantification. The comprehensive and automated quantification of radiomic features using data characterization algorithms^{25, 132, 133} can reflect biologic properties/tissue aberrations, for example, intra- and inter-organ tissue heterogeneities.¹³⁴ However, there is a lack of standardization of both feature definitions and image processing, which makes the reproduction and comparison of results challenging.¹³⁵ PyRadiomics 210 was developed to overcome this problem.²⁹ PyRadiomics enabled processing and quantification of radiomic features from medical imaging data through both simple and convenient front-end interface in 3D Slicer¹³⁶ and a back-end interface allowing automatic batch processing of the feature extraction. The reliability of implementing PyRadiomics to extract radiomic features from segmented regions of interest has been objectively proven.¹⁴ The definitions and interpretation of these features have been described previously.^{133, 137}

C.5.3. Study design—Aim 1. The conceptual overview of this aim is shown in FIG. 2 (C.1).

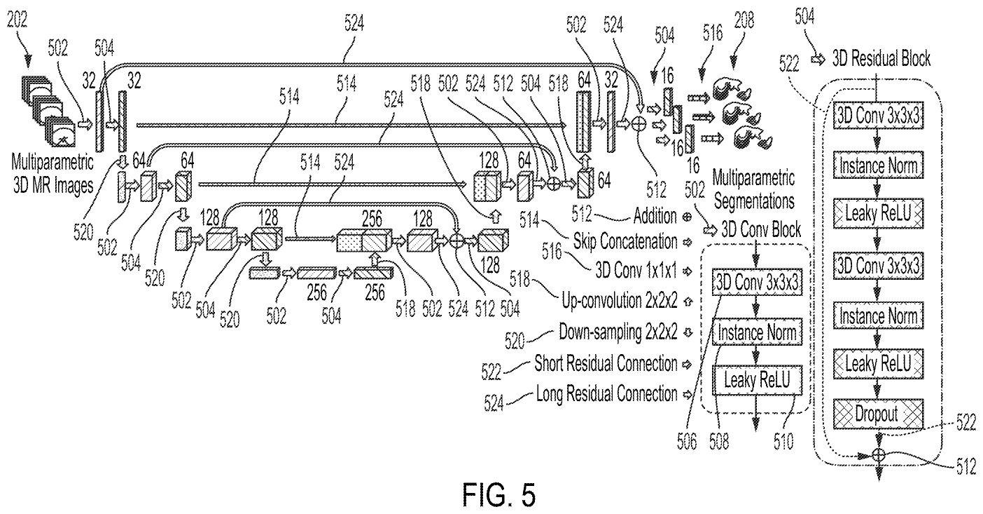

C.5.3.1. SLRes-U-Net model 206 design. FIG. 5 illustrates an architecture of the exemplary 3D SLRes-U-Net for multi-organ segmentation using multiparametric MRI data. The arrows denote different operations. The 3D boxes represent extracted feature maps, and their hash fillings are associated with the corresponding prior operations. Transparent boxes are copied feature maps. The number of convolutional filters feature channels is displayed on the top of each 3D box. The detailed layers of 3D convolutional and residual blocks are illustrated on the right.

In short, the exemplary novel SLRes-U-Net model 206 will be a special type of U-shaped CNN with both short and long residual connections to simultaneously take multiparametric MRI (e.g., T1-, T2-, and diffusion-weighted images) 202 as inputs and jointly segment liver and spleen 208. The network architecture of the exemplary SLRes-U-Net model is symmetric, having an encoder (FIG. 5, left side) that extracts spatial feature maps from the input images 202, and a decoder (FIG. 5, right side) that constructs the segmentation map from the encoded feature maps. To further detail the architecture of the exemplary LSRes-U-Net model, two terms are defined: 3-dimensional (3D) convolutional block (CB) 502 and 3D residual block (RB) 504. 3D CB (502) contains a 3D convolutional layer 506, an instance normalization layer 508¹³⁸, and a leaky rectified linear unit (ReLU) layer 510¹³⁹. The 3D convolutional layer 506 contains multiple convolution filters, each of which forms a feature channel. Compared to 3D CB, 3D RB (504) contains an additional short residual connection 522, linking the input with the output feature maps of the RB 504 and performing a summation operation 512. This short residual connection 522 not only maintains the spatial location information of the data across skipped network layers, but also smoothly propagates the error flow of model training backward within each level of encoder and decoder, improving the training efficiency and model performance. The encoder involves a sequence of 3D CBs 502 and 3D RBs 504. Inspired by the design of original U-net,⁷⁹ this sequence followed by a down-sampling operation 520 is repeated four times, and after down sampling operation at each level, the number of the feature channels will be doubled. On the contrary, the decoder, involving a succession of 3D CBs 502 and 3D RBs 504, up-samples 518 the feature maps and reduces the number of the feature channels by half at each successive level. At each successive level of the model, the feature maps of the encoder are transferred and concatenated to the feature maps of the corresponding decoder via a skip concatenation connection 514, which allows the model to retrieve the spatial information lost by pooling operations.¹⁴⁰ Besides the short residual, long residual connections 524 to connect CBs 502 with the same successive level in the encoder and decoder may be designed. The long residual connections 524 can propagate the spatial information from the encoder to the decoder to recover the spatial information loss caused by down-sampling operations 520 for more accurate segmentation. In addition, such design can more smoothly propagate the gradient flow backward through summation operations 512, and hence improve the training efficiency and network performance. In summary, both short 522 and long 524 residual connections can effectively propagate context and gradient information both forward and backward during the end-to-end training process. The final segmentations 208 may be generated by three parallel 3D convolutional layers with 1×1×1 filters 516. The number of feature channels of the first 3D CB 502 and the number of down-sampling operations 520 are optimizable hyperparameters.

C.5.3.2. Radiomic and deep features extraction. Based on the liver and spleen segmentations, embodiments may run a well-established PyRadiomics pipeline 210 to extract radiomic features 212.²⁹ Radiomic features may include 13 geometric features (e.g., surface area, compactness, maximum/minimum diameters, sphericity), 18 histogram (first-order) features (e.g., variance, skewness, kurtosis, uniformity, entropy), 14 texture features from the gray-level dependence matrix, 23 texture features from the gray-level co-occurrence matrix, 16 texture features from the gray-level run-length matrix, 16 texture features from the gray-level size zone matrix, and five texture features from the neighborhood gray-tone difference matrix. Embodiments may implement a pre-trained very deep CNN 214 with fixed hyperparameters, but without its original classifier, to extract deep features (C.4.4).

C.6. Aim 2 (218). An “ensemble” DL model (LFNet) 220 to predict biopsy-derived LF stage and LF percentage 222 using the integration of multiparametric MRI radiomic 212 and deep features 216, MRE 204, and routinely-available clinical data 224.

C.6.1. Rationale. Different causes of CLD (e.g., NASH, viral hepatitis, metabolic, cholestatic disease, cardiac disease) may all lead to LF, which is characterized by the excessive accumulation of collagen and extracellular matrix.^{141, 142} Accurate diagnosis and quantification of LF is vital, as it is prognostic and informs medical and surgical decision-making. Although liver biopsy is the current standard for assessing LF, it is prone to sampling error and invasive with low patient acceptance.¹⁴³⁻¹⁴⁵ MRI with MRE represents the latest technology for diagnosis and characterization of LF and the overall assessment of CLD.¹⁴⁶⁻¹⁴⁸ In contrast to ultrasound and computed tomography techniques, MRI provides superior soft tissue contrast and permits repeated assessments without ionizing radiation concerns. MRI-based radiomic features related to signal intensity, morphology, and texture of the liver and spleen have been reported useful for detection of LF.^{146, 149-153} Multiple liver MRI sequences have been investigated for radiomic analysis, including T1-weighted,¹⁵⁴ T2-weighted,¹⁵² proton density-weighted,¹⁵⁵ and DWI.¹⁵⁶⁻¹⁵⁸ Various computer-aided models (e.g., classical statistical analysis, conventional ML, and the state-of-the-art DL) have been developed to quantitatively analyze MRI features and facilitate the diagnosis of LF, but none of these is sufficiently accurate.^{115, 147, 151, 152, 156, 159-163} Each prediction model has its own strengths and weaknesses, and it therefore is natural to expect that a learning method that takes advantages of multiple prediction models would lead to superior performance. To this end, embodiments employ a stacking “ensemble” learning technique aims to integrate multiple models to fill in each other's weaknesses, thereby rendering better diagnostic performance over each individual one.³⁷ The intuitive explanation of why stacking ensemble learning works is from human nature and seeking the wisdom of crowds in making a complex decision. Theoretically, the reasons to explain why stacking ensemble learning works include overfitting avoidance, computational efficiency, and hypothesis enforcement.^{164, 165} In the last decade, model stacking has been successfully used on a wide variety of predictive modeling problems to boost the models' prediction accuracy beyond the level obtained by any of the individual models. More recently, there have been attempts to develop DL ensemble models.³⁸⁻⁴² It has been noted that in data science competitions (global challenges to produce the best model for a specified performance criterion based on the issued training and test data), the winning model is most commonly an ensemble model.¹⁶⁶ This disclosure provides an exemplary DL ensemble model (LFNet) 220 that may quantify biopsy-derived LF stage and LF percentage 222 using the integration of multiparametric MRI radiomic 212 and deep features 216, MRE-derived LS, and routinely-available clinical data 224.

C.6.2. Preliminary Studies.

Diagnostic performance of MRE to quantify LF. MRE has demonstrated variable diagnostic performance based on the literature. A retrospective study⁸ by our group included 86 pediatric patients (49 [57%] boys; median age=14.2 years [range, 0.3-20.6 years]) who underwent MRE and liver biopsy within 3 months of one another for indications other than liver transplantation or Fontan palliation. The AUROC for LF stage 0-1 versus stage 2 or higher fibrosis was only 0.70 (95% CI: 0.59, 0.81) for the whole population, and was significantly lower for patients with steatosis versus those without (AUROC: 0.53 [95% CI: 0.35, 0.71] vs. 0.82 [95% CI: 0.67, 0.96]; p=0.01). The optimal LS cut-off value for the entire population was 2.27 kPa, with 68.6% sensitivity (95% CI: 57.2%, 80.1%) and 74.3% specificity (95% CI: 63.5%, 85.1%). These results suggest that MRE has only moderate diagnostic performance in children and that there may be a confounding effect of steatosis or inflammation in the NAFLD/NASH population. In a study of 289 adult patients from the Mayo clinic that underwent MRE within one year of biopsy, LS was shown to increase with increase LF.¹⁶⁷ However, close inspection of the error bars shows considerable overlap of LS for all LF stages.¹⁶⁷ A recent study by Furlan et al. in adult NAFLD/NASH patients demonstrated an MRE AUROC of 0.85 (95% CI: 0.74, 0.95) for identifying significant fibrosis (F0-1 vs. F2-4).¹⁶⁸

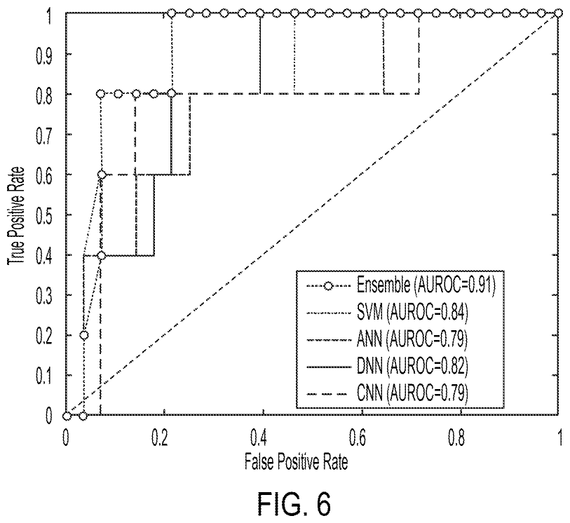

Ensemble learning model to improve the prediction performance. Embodiments demonstrated improved performance by using a stacking ensemble learning approach in early prediction of cognitive deficits in a preterm cohort study. A two-level ensemble model has been developed. On the first-level, four different prediction models were trained, including a SVM classifier on the volume quantifications of white matter abnormality, an ANN classifier on clinical risk factors, a transfer learning enhanced deep neural network (DNN) classifier on imaging features derived from functional MRI, and a transfer learning enhanced CNN classifier on imaging features derived from diffusion tensor MRI. On the second-level, an SVM model was used to fuse the prediction probabilities from all four models to generate a final prediction. The results (FIG. 6) showed that the ensemble model overperformed each of the individual models, achieving an accuracy of 81.8% and AUROC of 0.91 on the classification of patients into high-risk versus low-risk of developing cognitive deficits. As shown in FIG. 6, the area under the receiver operating characteristic curve (AUROC) of the ensemble model outperformed those of individual models for early prediction of cognitive deficits.

C.6.3. Study design—Aim 2 (218). The conceptual overview of this aim 218 is shown in FIG. 2 (C.1).

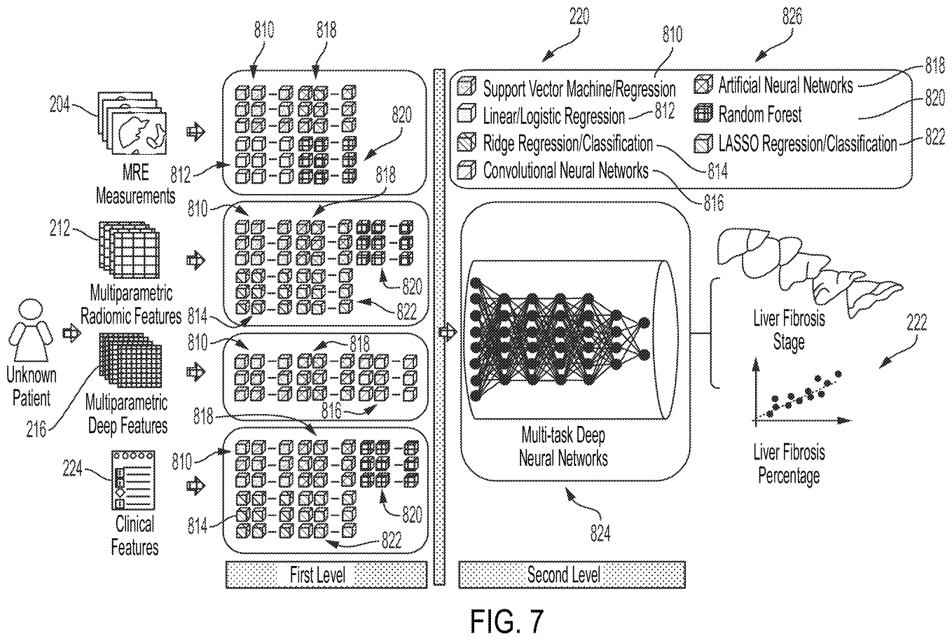

C.6.3.1. LFNet model (220) design. LFNet 220 is designed in an embodiment to be a two-level ensemble model (FIG. 7), combining the predictive power of both state-of-the-art DL and traditional ML. FIG. 7 is a block diagram illustrating architecture of an exemplary ensemble LFNet model 220 for liver fibrosis prediction 222. Each input data type (MRE-derived LS 204, multiparametric MRI radiomic 212 and deep features 216, and routinely-available clinical data 224) may be used to create multiple unique ML models (810, 812, 814, 816, 818, 820 & 822). The output of these models is then integrated using a multi-task deep neural network 824. The output 222 of the deep neural network 824 will include both predicted histologic liver fibrosis stage (F0-F4) and fibrosis percentage (0-100%).

1) First, a diverse model library is built. The diversity plays a key role, and it is a necessary and sufficient condition in building a powerful stacking ensemble model.^{165, 169, 170} Each of input data types (MRE-derived LS 204, multiparametric MRI radiomic 212 and deep features 216, and routinely-available clinical data 224) may be used to create multiple unique ML models (810, 812, 814, 816, 818, 820 & 822). The model library 826 that may consist of a diverse set of multiple traditional ML models, including SVM (810),⁴⁵ ANN (818),³⁰ random forest (820),⁴⁴ logistic regression (812),⁴³ Ridge (814)¹⁷¹ and least absolute shrinkage and selection operator (LASSO) (822).¹⁷² Multiple same type of models may be trained with different hyperparameter settings and training datasets; and then 2) the multiple ML classifiers from the model library 826 are integrated using a DL model. Multi-channel, multi-task DNN 824 may be applied as a fusion model. The number of channels may be designed based on the number of models in model library 826. Each input channel may contain several neural network blocks. The multiple input channels may be eventually fused into one output channel through a fusion block. Each block may include a fully-connected layer, a batch normalization layer, and a dropout regularization layer. Followed by the fusion block, a softmax output layer may be used to predict fibrosis stage (F0-4); and a linear regression layer may be used to quantify fibrosis percentage (0-100%).

C.7. Aim 3 (226). A DL model (LSNet) 228 to quantify MRE-derived LS 230 using multiparametric MRI radiomic 212 and deep features 216 as well as clinical features 224.

C.7.1. Rationale. MRE is increasingly used for detecting and assessing the severity of CLD in children and adults.¹⁷³ MRE involves the generation of liver transverse (shear) waves using an active-passive driver system (the passive driver is placed over the right upper liver). These waves and associated displacement of liver tissue can be imaged using a modified phase-contrast pulse sequence and can be used to create quantitative images of LS.^{174, 175} MRE is currently used as a surrogate biomarker for LF.¹⁷⁶⁻¹⁷⁸ Although RE obviates the need for liver biopsy in some patients and allows more frequent longitudinal monitoring of liver health, it has associated drawbacks related to additional patient time in the scanner, patient discomfort, and added costs (e.g., infrastructure and patient charge-related); the cost of adding MRE to a given MRI scanner is ˜$100,000-250,000 in the U.S. for necessary hardware and software purchases. We have previously developed a SVM model to categorically classify MRE-derived LS (<3 vs. ≥3 kPa) using only readily-available clinical and non-elastographic T2-weighted MRI radiomic features in pediatric and young adult patients with known or suspected liver disease.¹⁴ More recently, we also have demonstrated the feasibility of creating a DL model for the same purpose. Both the SVM and DL models showed similar fair-to-good-diagnostic performance and have the potential to facilitate the identification of patients with likely normal or near-normal LS for whom MRE may not be indicated.

In embodiments of the current disclosure, instead of categorical classification, a DL regression model is provided to predict continuous MRE-derived shear LS (˜1-12 kPa). Such an algorithm could direct and/or even eliminate the use of MRE, thereby decreasing imaging time and saving considerable healthcare costs (likely 10s of millions of U.S. dollars yearly).

C.7.2. Preliminary Studies.

LS classification using ML on T2-weighted MRI radiomic features.¹⁴ We included 309 patients with known or suspected CLD in this retrospective study. For each patient, we extracted 105 radiomic features from T2-weighted fat-suppressed fast spin-echo images. The number of radiomic was reduced to prevent model overfitting using a LASSO algorithm.¹⁷² A SVM⁴⁵ model then was used to conduct two-class classification. An exemplary model was built and internally validated using 225 unique examinations. A leave-one-out cross-validation strategy was used to estimate the diagnostic performance of classifying LS<3 vs. ≥3 kPa. Our internal cross-validation shows an AUROC of 0.70 using radiomic features for the classification, and an AUROC of 0.84 when combined with clinical features. In our external validation experiment, this SVM model achieved an AUROC of 0.80. Two highly discriminative features in our combined radiomic and clinical model related to radiomic liver texture. The fact that texture features are important makes intuitive sense, because more normal-appearing liver tissue generally appears relatively hypointense and homogeneous, and the liver becomes increasingly hyperintense and heterogeneous with worsening parenchymal fibrosis.

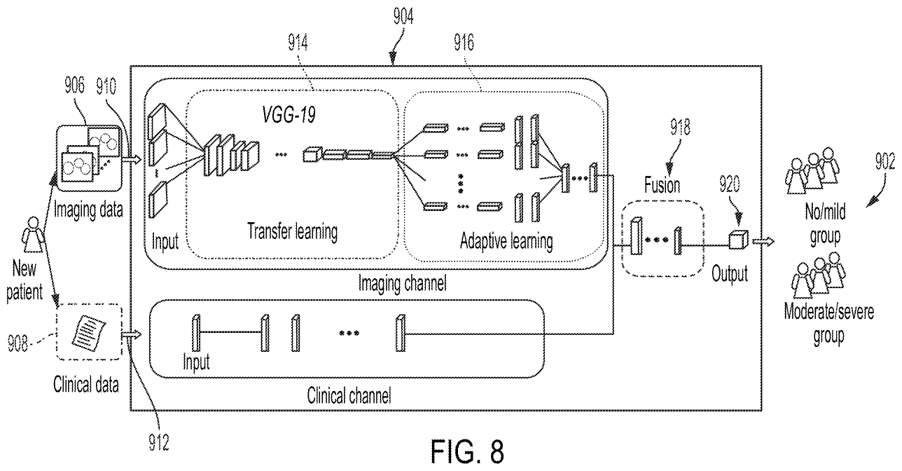

LS classification using DL on T2-weighted MRI deep features.¹⁸ In this recent work from our group, we included 273 patents with known or suspected CLD. An exemplary DeepLiverNet (FIG. 8) was used to classify a given patient into one of two groups: no/mild (<3 kPa) vs. moderate/severe (≥3 kPa) liver stiffening. As illustrated in FIG. 8, Liver stiffness stratification 902 was obtained with DeepLiverNet 904 using anatomical axial T2-weighted fast spine-echo fat suppressed MR images 906 and clinical data 908. Such outputs 902 may be communicated to the user via computer display, electronic messaging, print-out, or any other known mechanism for communication.

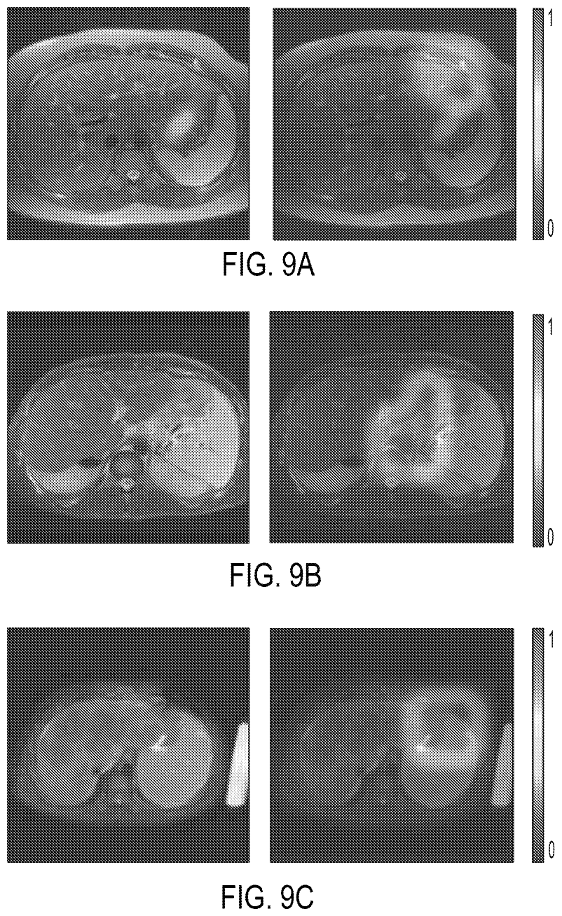

DeepLiverNet contained two separate input channels 910, 912 for imaging 906 and clinical data 908, respectively. For the imaging channel 910, transfer learning layers 914 were first designed by reusing a pre-trained very deep CNN model (VGG-19) for T2-weighted MRI deep feature extraction. It was followed by adaptive learning layers 916 to learn the latent imaging features unique to the severity of LS. The clinical channel 912 was designed to capture the latent clinical features. Then, fusion layers 918 were employed to integrate the latent imaging and clinical features. Lastly, a softmax classifier 920 was used to predict the outcome. The DL model was trained using a stochastic gradient descent algorithm. Rotation and shift-based data augmentation methods were utilized to enlarge the training samples by 10 times. Internal 10-fold cross-validation with 178 examinations shows an AUROC of 0.80 (95% CI: 0.79, 0.81) using deep features and AUROC of 0.86 (95% CI: 0.85, 0.87) when combined with clinical features. External validation of the DL model with an independent dataset consisting of 95 MRI examinations achieved an AUROC of 0.77. Saliency maps (Grad-CAM)^{70, 72} also were created to show areas of deep feature discrimination (FIGS. 9A-C provide saliency maps showing areas of greatest deep feature discrimination).

Further discussion of DeepLiverNet 904 is provided below in Section E.

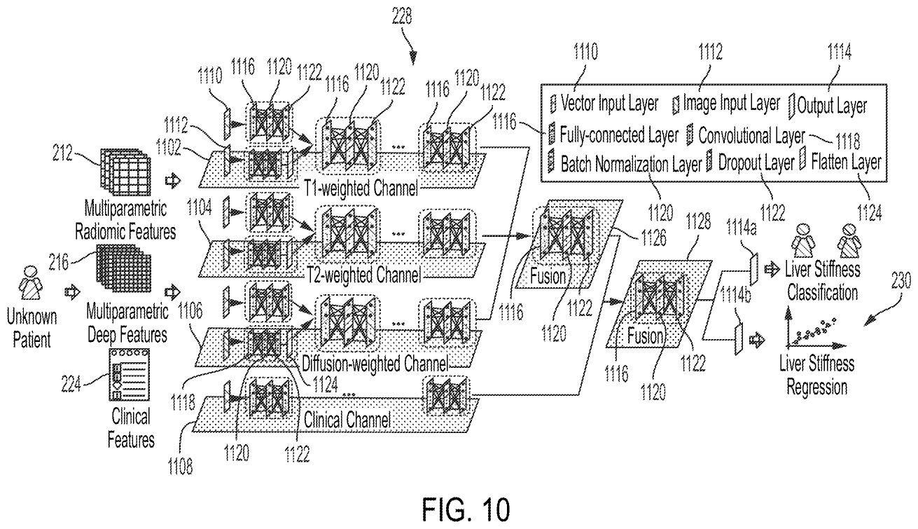

C.7.3.1. LSNet model design. FIG. 10 provides architecture of the exemplary LSNet model 228 for liver stiffness quantification 230. Such outputs 230 may be communicated to the user via computer display, electronic messaging, print-out, or any other known mechanism for communication. LSNet 228 is a multi-channel multi-task DL model that uses multiparametric radiomic 212 and deep features 216 as well as clinical data 224 as inputs, and that can classify a given patient into one of two groups (e.g., no/mild vs. moderate/severe [≥3 kPa] liver stiffening) as well as predict his/her (kPa). As shown in FIG. 10, exemplary LSNet 228 includes four input channels, including three imaging channels (T1-weighted (1102), T2-weighted (1104), diffusion-weighted (1106)) and one clinical channel 1108. Each imaging channel 1102, 1104 & 1106 further includes two subchannels, for radiomic and deep features respectively. Radiomic subchannel contains an input layer 1110 handling one-dimensional radiomic feature vector, a fully-connected layer 1116, a batch normalization layer 1120, and a dropout layer 1122. Deep subchannel contains an input layer 1112 handling two-dimensional deep feature maps, a convolutional layer 1118, a batch normalization layer 1120, a dropout layer 1122, and a flatten layer 1124. The structure of clinical channel 1108 may be same as the radiomic subchannel, as both radiomic and clinical features can be vectorized. The radiomic and deep subchannels may be fused 1126 to summarize the latent information from all imaging data; this output may be further fused 1128 with latent information from clinical data. Embodiments may have a softmax output layer 1114a for LS classification and a linear regression output layer 114b for LS prediction (kPa).

D. Summary. Embodiments may result in internally and externally validated prognostic models for quantifying LF and LS. Exemplary DL techniques may be employed for the prediction of other important clinical outcomes in CLD (inflammation, onset of portal hypertension and related complications, time to transplant/transplant listing, mortality, etc.) as well as to other organs and diseases.

E. DeepLiverNet (904)—A machine learning model that can categorically classify the severity of liver stiffening using both anatomic T2-weighted MR images and clinical data for pediatric and young adult patients with known or suspected chronic liver disease

E.1.a Although magnetic resonance elastography (MRE) allows quantitative evaluation of liver stiffness to assess chronic liver diseases, it has associated drawbacks related to additional scanning time, patient discomfort, and added cost.

E.1.b Population: In an IRB-approved retrospective study, we included 273 subjects with known or suspected chronic liver disease that had undergone liver MRE.

Sequence: Axial T2-weighted fast spin-echo fat-suppressed MR images, pertinent clinical data, and MRE liver stiffness measurements were extracted from our Picture Archiving and Communication System (PACS) and electronic medical record system.

E.1.c Assessment: DeepLiverNet 904 is an exemplary multi-channel deep transfer learning convolutional neural networks to classify a patient into one of two groups: no/mild vs. moderate/severe liver stiffening (<3 kPa vs. ≥3 kPa) 902. Internal cross-validation and external validation were conducted. Diagnostic performance was assessed using accuracy, sensitivity, specificity, and area under the receiver operating characteristic curve (AuROC).

E.1.d Statistical Analysis: The two-sided student's t-test and chi-squared test were used to assess baseline differences between cohorts and models' performance.

E.1.e Results: In the internal cross-validation, the combination of clinical and imaging data produced the best performance (AuROC=0.86) compared to clinical (AuROC=0.83) or imaging (AuROC=0.80) data alone. Using both clinical and imaging data, the DeepLiverNet correctly classified patients with accuracy=88.0%, sensitivity=74.3%, and specificity=94.6%. In the external validation, this same deep learning model achieved an accuracy=80.0%, sensitivity=61.1%, specificity=91.5%, and AuROC=0.77.

E.1.f Data Conclusion: A deep learning model that incorporates clinical data and anatomic T2-weighted MR images may provide a means of risk stratifying liver stiffness and directing the use of MRE, potentially eliminating its use in some patients.

E.2 DeepLiverNet Introduction

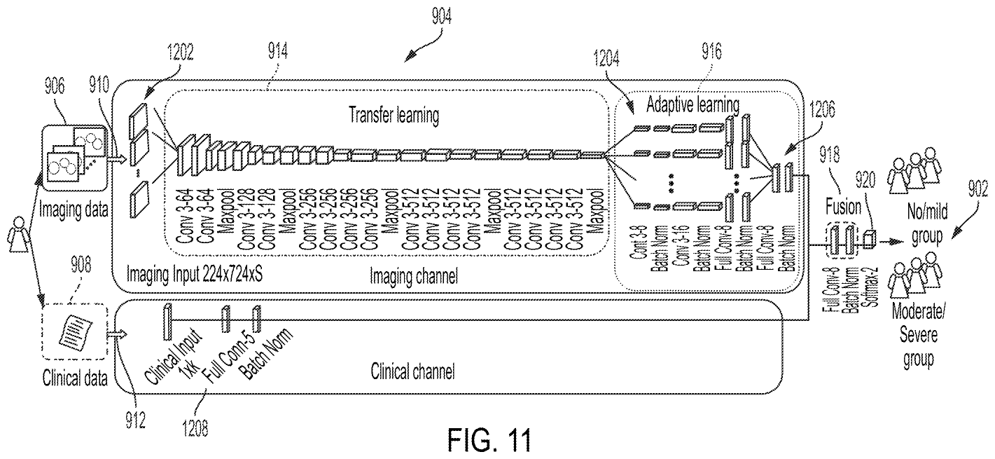

A deep learning approach has been developed to classify the severity of liver stiffness as determined by MRE using clinical features and anatomic MR imaging data (FIG. 11). Specifically, DeepLiverNet, a multi-channel deep transfer learning convolutional neural network classification model, is provided to categorically classify MRE-derived liver stiffness by integrating clinically-available features and axial T2-weighted fat-suppressed MR structural liver images in pediatric and young adult patients with known or suspected chronic liver disease. Transfer learning and data augmentation may be utilized to aid model training. DeepLiverNet was comprehensively evaluated using internal cross-validation and also external validation on an independent cohort.

E.3 Materials and Methods

Department of Radiology records were searched from January 2011 through October 2018 to retrieve clinically performed MRE examinations, irrespective of clinical indication or patient age. Two cohorts (internal validation cohort and external validation cohort, respectively) were identified. The internal validation cohort scanned on 1.5T and 3T GE Healthcare MRI scanners (Waukesha, Wis.) was used for model development and internal validation. The external validation cohort was scanned on 1.5T and 3T Philips Healthcare MRI scanners (Best, the Netherlands). Only one MRE examination was selected from each unique patient (the most recent), with the other MRE examinations excluded. Examinations from patients with missing clinical and imaging data were excluded. Ultimately, this resulted in 178 MRE examinations for the internal validation cohort and 95 MRE examinations for the external validation cohort.

The institutional MRE technique used during the study period has been described in prior publications.²⁰³ The mean liver stiffness value (mean of four anatomic levels/slices through the mid liver, weighted for region-of-interest [ROI] size) in kPa (shear modulus) was retrieved from the clinical imaging report of each MRE examination. Based on mean liver stiffness, patients were divided into two groups (<3 kPa=no/mild vs. ≥3 kPa=moderate/severe liver stiffening). A cut-off value of 3 kPa was chosen as it provides both reasonable clinical sensitivity and specificity for detecting abnormal liver stiffening based on the literature in both pediatric and adult cohorts as well as our prior support vector machine classifier work.^{191,204-206} Liver volume in mL, liver chemical shift-encoded fat fraction (%), presence of liver fat (fat fraction >5%), and MRI scanner information (i.e., manufacturer, machine model, field strength) also were extracted from clinical imaging reports.

E.4 T2-Weighted MR Images

Axial two-dimensional T2-weighted fast spin-echo fat-suppressed images that were obtained as part of routine clinical MRE examination were extracted from our clinical Picture Archiving and Communicating System (PACS). T2-weighted images were obtained using the following parameters/parameter ranges during the study period: TE=˜85 ms; TR=>3000 ms; flip angle=90 degrees; number of signal averages=2; parallel imaging acceleration factor=2; matrix=˜256×224; and slice thickness=5-6 mm. Individual T2-weighted images were normalized to a field of view of 300×300 mm², with an in-plane resolution 1.0×1.0 mm.

E.5 Clinical Data

For each subject, 27 clinical features were retrieved from the electronic medical record system (Epic Systems Corporation; Verona, Wis.), with only those values/records within six months of the MRE examination. Clinical data from three major domains were obtained: 1) demographic/anthropomorphic data (e.g., age, sex, body mass index); 2) medical history/diagnoses (e.g., diabetic status, specific diagnoses, such as non-alcoholic fatty liver disease, viral hepatitis, or primary sclerosing cholangitis); and 3) laboratory testing (e.g., alanine aminotransferase, aspartate aminotransferase, bilirubin, albumin, gamma-glutamyl transferase, and Fibrosis-4 score). The list of clinical features used for developing the exemplary model is as follows.

List of non-deep imaging features and MRI scanner information from MR elastography examinations of all patients that were included in exemplary models. These features were combined with axial T2-weighted MR images as imaging data to train the DeepLiverNet.

Extracted Non-Deep Features from Standardized Imaging Reports
Liver volume (ml)
Liver chemical shift-encoded fat fraction (%)
Presence of liver fat (chemical shift-encoded fat fraction >5%)

MRI Scanner Information
Scanner manufacturer
Scanner model
Scanner field strength

List of clinical features obtained from all patients and used in the clinical input channel of our models.
Age
Sex
Race
Height
Weight
Body mass index (BMI)
Systolic blood pressure
Diastolic blood pressure
Diabetes mellitus, type 1 (yes or no)
Diabetes mellitus, type 2 (yes or no)
Non-alcoholic fatty liver disease (including non-alcoholic steatohepatitis) (yes or no)
Fontan operation (yes or no)
Biliary atresia/biliary atresia status post Kasai portoenterostomy (yes or no)
Primary sclerosing cholangitis (yes or no)
Autoimmune hepatitis (yes or no)
Cystic fibrosis (yes or no)
Alagille syndrome (yes or no)
Alanine transaminase (ALT)
Aspartate transaminase (AST)
Gamma-glutamyl transferase (GGT)
Total bilirubin
Direct bilirubin
Alkaline phosphatase
Platelet count
Albumin
Fibrosis-4 (FIB-4) score
AST to platelet ratio index (APRI)

E.6 Overview of Liver Stiffness Stratification

An exemplary task is to classify a given patient with known or suspected chronic liver disease into one of two groups 902: no/mild liver stiffening vs. moderate/severe liver stiffening (See FIG. 11).

E.6.a Architecture of DeepLiverNet

FIG. 11 provides a diagram of an exemplary model of DeepLiverNet 904. The exemplary model contains two separate input channels 910, 912 for imaging and clinical data, respectively. For the imaging channel, a transfer learning block 914 was designed by reusing a pre-trained deep model for image feature extraction. It was followed by an adaptive learning block 916 to learn the latent imaging features unique to indicating the presence of liver stiffening. The clinical channel 912 was designed to capture the latent clinical features. Then, a fusion block 918 was employed to integrate the latent imaging and clinical features. Lastly, a softmax classifier 920 was used to stratify the severity of liver stiffness 902.

A multi-channel (i.e., imaging channel and clinical channel) deep architecture was utilized in our DeepLiverNet to take individual axial 2D T2-weighted MR images (e.g., S slices of images) and clinical data (e.g., k clinical features), simultaneously.

The imaging channel 910 is comprised of an image input layer 1202, a transfer learning block 914, and an adaptive learning block 916. First, the image input layer 1202 contains S parallel input sub-channels, taking S number of individual slices of fixed-size axial T2-weighted MR images. Next, to extract liver image features, the transfer learning block 914 is designed by reusing available pre-trained deep models. We chose to reuse the weights of the VGG-19 model²⁰⁷ (from 1^{st} to 21^{st} layers) for the transfer learning block 914. Then, we designed the adaptive learning block 916 that contains S parallel sub-channels 1204 corresponding to the input sub-channels for learning the individual latent features of S liver slices, respectively. At the end, those sub-channels 1204 in the adaptive learning block are integrated by a fully-connected layer 1206.

For the clinical channel 912, a fully-connected layer 1208 is directly applied to learn the latent features from the clinical data represented by a low-dimension vector (e.g., k features). After the feature extraction, a fusion block 918 is applied to integrate the latent features from both imaging and clinical data. A two-way softmax classifier 920 was utilized to classify the severity of liver stiffness 902.

The exemplary architecture design was based on brute-force searching the space (i.e., limited combinations of the numbers of layers and neurons). For the adaptive learning block and clinical channel, we tested the number of neurons from empirical values.^{186,194,210,212} The size of convolutional filters was set as 3×3 as suggested in VGG model design.²⁰⁷ In addition, multiple publicly available pre-trained deep ImageNet models (based on ˜1.2 million color images) (http://www.image-net.org/) were tested. The candidate ImageNet models that we compared included VGG-16 and VGG-19 models,²⁰⁷ ResNet,²⁰⁸ Inception,²⁰⁹ and NASNet,²¹⁰. We divided the interval validation cohort into training (80%), validating (10%), and testing data (10%). Various combinations of the architecture options were tested, and the one with the best performance on the validating dataset was considered optimal for this study.

Referring again to FIG. 11, liver stiffness stratification with DeepLiverNet 904 using anatomic two-dimensional axial T2-weighted fast spin-echo fat-suppressed MR images 906 and clinical data 912. The input of the imaging channel is S of axial 2D T2-weighted MR images with a size of ˜256×224, and the input of the clinical channel is a vector of clinical features. The type of layers, the size of filter, and the number of neurons were listed for individual layers. Cony: Convolutional layer; Maxpool: Maxpooling layers; Batch Norm: Batch normalization layer; Full Conn: Fully-connected layer. In an embodiment, the transfer learning 914 layers are non-trainable layers, while other layers are trainable. For example, Conv3-64 means a convolutional layer with 64 convolutional neurons (filter size: 3×3)

E.6.b Training of DeepLiverNet

Let ([Xᵢⱼ^{I}]ⱼ₌₁^{S},xᵢ^{C},yᵢ)ᵢ₌₁^{N} denote a training sample set with N subjects. For the i^{th} subject, [Xᵢⱼ^{I}]ⱼ₌₁^{S} is the j^{th} slice of imaging data with a total of S liver slices, xᵢ^{C} is the clinical data, and yᵢ is the severity group label. Imaging and clinical data as well as the associated group labels are utilized in a back-propagation procedure to train the proposed DeepLiverNet. To train the deep model, the cross-entropy loss function is defined by:

$$ℒ ( W , b ) = - \frac{1}{N} {\sum }_{i = 1}^{N} {y}_{i} log ( p ( {y}_{i} \vert {[ {X}_{i j}^{I} ]}_{j = 1}^{S} , {𝓍}_{i}^{C} ; W , b ) ) + ( 1 - {y}_{i} ) log ( 1 - p ( {y}_{i} \vert {[ {X}_{i j}^{I} ]}_{j = 1}^{S} , {𝓍}_{i}^{C} ; W , b ) ) \qquad ( Equation 1 )$$

Where p(yᵢ|[Xᵢⱼ^{I}]ⱼ₌₁^{S}, xᵢ^{C}; W, b) is the probability of the i^{th} subject being classified as a positive class. The above loss function was minimized by a mini-batch Adam algorithm²¹¹ so as to optimize the weights W and bias b of DeepLiverNet. The mini-batch strategy divided the training data into m batches and updates the model m times in each training epoch, enabling a fast and stable convergence. A batch size of 16 was selected from empirical values.^{186,194,210,212} The learning rate was set as 0.01 after testing several empirical values [0.001, 0.01, 0.1, 0.5]. Batch size and learning rate were chosen based on successful convergence of model training. To further accelerate the model convergence, we applied a gradient update decay parameter as 0.0003 (learning rate/maximal epoch). The number of epochs was set as 30. We applied an early stop mechanism, which would cease the optimization process if 5 consecutive epochs return the same validation loss errors. The proposed DeepLiverNet was implemented by Python 3.6, Keras (version: 2.2.4) with Tensorflow (version: 1.10) backend on a computer workstation (256 RAM, 2×NVIDIA GTX1080 Ti with CUDA 10.0).

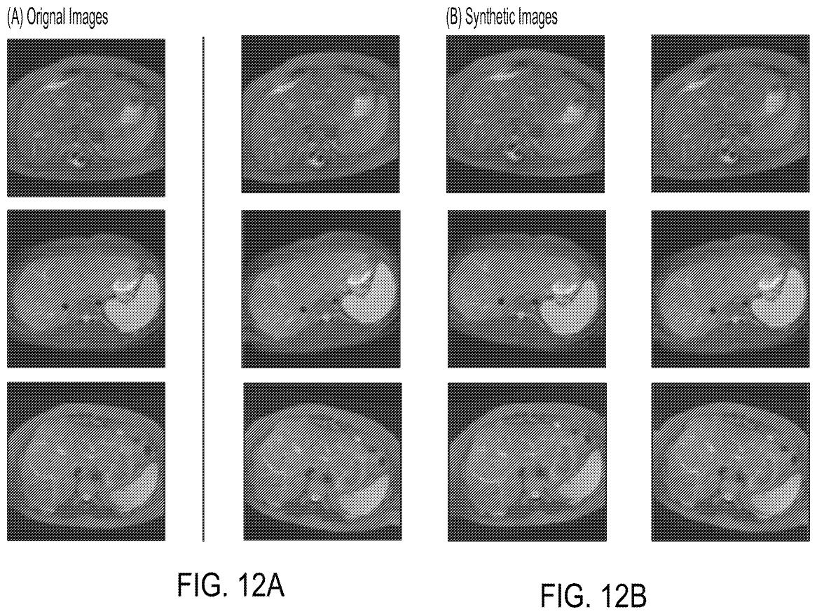

Due to the limited sample size and slightly imbalanced subject ratio (i.e., <3 vs. ≥3 kPa=˜2:1 in the current study), a rotation and shift-based data augmentation scheme²¹² is used to increase the training data and balance subject ratio. Augmentation includes random image rotation (≤10°) as well as vertical and horizontal shifting (≤5 voxels) on a randomly selected T2-weighted image. FIGS. 12A & B respectively illustrate the original liver images (FIG. 12A) and three randomly synthesized liver images (FIG. 12B) from three different subjects. The process was firstly repeated until the number of subjects were equal in two groups. We then augmented the training samples by 10 times, while the testing dataset of any experiment is fully excluded from data augmentation procedures.

Referring to FIGS. 12A & B, original axial T2-weighted MRI liver images (FIG. 12A) and three randomly synthesized MRI liver images (FIG. 12B) using the rotation and shift-based data augmentation algorithm are provided. Each row is an axial 2D slice of T2-weighted MRI liver images from a randomly selected subject. A random image rotation (≤10°) and a random vertical and/or horizontal shifting (≤5 voxels) were applied on the original images.

E.7.a Internal Validation (1410)

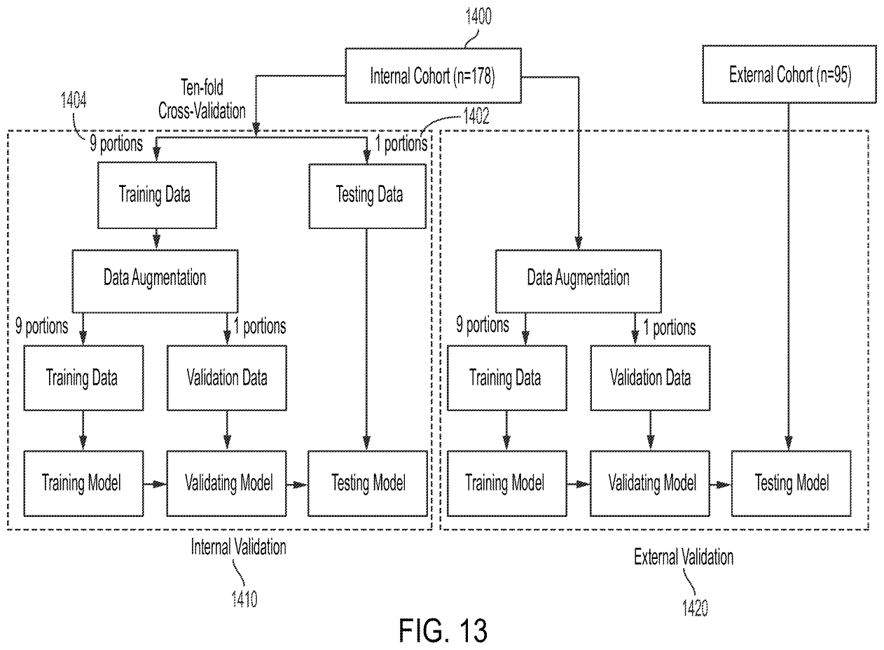

Referring to FIG. 13, we developed and validated our deep model using the internal validation cohort (178 unique examinations from patients scanned with MRI scanners manufactured by GE Healthcare) 1400. Clinical MRE examinations obtained for this study contained axial images (6.5 mm slice thickness) sampled through the liver volume. Individual T2-weighted slices corresponding to those MRE anatomic slice levels were identified, i.e., S=4. Subject-wise 10-fold cross-validation was used to test the DeepLiverNet. In each iteration of the 10-fold cross-validation, the subjects in the whole cohort were divided into 10 portions of approximately equal size. One portion of cohort 1402 was utilized for testing, while the rest nine portions of cohort 1404 were used for model training. In addition, 10% of training data was treated as validating data to test the convergence of model training. We conducted this process 10 times until all 10 portions of cohort have been tested once. We then computed the average performance across all 10 times. To test the reproducibility of the model, we repeated such ten-fold cross-validation experiment 10 times and calculated the 95% confidence interval. The diagnostic performance of the model was assessed using the metrics of accuracy, sensitivity, specificity, and area under the receiver operating characteristic curve (AuROC).

FIG. 13 provides internal and external validation experiments flow chart. In the external validation, we trained our DeepLiverNet using 178 patients from our internal validation cohort and tested the model using 95 unseen subjects from the external validation cohort

E.7.b External Validation (1420)

The DeepLiverNet was externally validated by using examinations from an independent cohort of 95 unique patients scanned on MRI scanners manufactured by Philips Healthcare. By testing the model on data collected from different manufacturer scanners, we are able to show the generalizability of the model when it is used as an off-the-shelf product on the unseen data. This is especially useful for the future potential clinic usage of the model when training the model with data from a particular scanner is not feasible. We trained our DeepLiverNet using 178 subjects from our internal validation cohort and tested the model using 95 unseen subjects from the external validation cohort. The same rotation and shift-based data augmentation methodology used in our internal validation experiment was applied to balance and augment the imaging data in our external validation experiment. Again, the diagnostic performance of the model was assessed using the metrics of accuracy, sensitivity, specificity, and AuROC.

E.7.c Statistical Analysis

Continuous data were summarized as means and standard deviations; categorical data were summarized as counts and percentages. The two-sided student's t-test (continuous data) and chi-squared test (categorical data) were used to assess baseline differences between cohorts and model performance. A p-value <0.05 was considered statistically significant for inference testing. Analyses were performed with the statistical package of Matlab 2018a (MathWorks, Natick Mass., United States).

E.7.d Results

No significant baseline differences were found between patients in our internal and external validation cohorts (Table 1).

TABLE 1
Baseline characteristics of internal and external validation cohorts.

| | Internal cohort | | External cohort | | p-value |
|---|---|---|---|---|---|
| MRI scanner | GE Healthcare | | Philips Healthcare | | — |
| manufacturer | | | | | |
| Number of subjects (n) | 178 | | 95 | | — |
| Age (years) | 14.7 | (4.8) | 14.0 | (5.3) | 0.29 |
| Male, number (%) | 117 | (65.7%) | 59 | (62.1%) | 0.55 |
| Body mass | 30.0 | (9.7) | 28.9 | (10.9) | 0.37 |
| index (kg/m²) | | | | | |
| Liver stiffness (kPa) | 2.9 | (1.1) | 3.1 | (1.4) | 0.11 |

Age, body mass index, and liver stiffness are presented as mean (standard deviation): Sex is presented as the number (percentage) of male patients.

E.8.a Internal Validation

One-hundred-and-seventy-eight MRE examinations performed on a GE Healthcare MRI scanner from 178 unique patients were used for internal validation experiment. One-hundred-and-twenty-one patients with a mean liver stiffness <3 kPa had a mean age of 14.2 (4.6) years; 85/121 (70.0%) patients were male. Fifty-seven patients with a mean liver stiffness ≥3 kPa had a mean age of 15.8 (5.0) years; 25/57 (56.1%) patients were male. There was no significant difference in age (p=0.05) or sex (p=0.06) between groups. Patients with a mean liver stiffness <3 kPa had a mean liver stiffness of 2.3 (0.4) kPa, while patients with a mean liver stiffness ≥3 kPa had a mean liver stiffness of 4.0 (1.2) kPa. One-hundred-and-forty-one (79.2%) MRE examinations were performed on 1.5T MRI scanners, and 37 (20.8%) MRE examinations were performed on 3T MRI scanners.

E.8.b Classifying Liver Stiffness Using T2-Weighted Imaging Data Alone

We first set to determine the performance of DeepLiverNet using only non-stiffness T2-weighted imaging data. Our DeepLiverNet was able to correctly classify patients with regard to categorical MRE liver stiffness with an AuROC of 0.80 (Table 2). The model, with imaging data only, achieved an accuracy of 85.2%, with a sensitivity of 66.1% and specificity of 93.0%.

TABLE 2
Diagnostic performance of DeepLiverNet model at validation for categorically classifying
patients using, imaging data alone, clinical data alone and combined data (n = 178).

| | Accuracy | Sensitivity | Specificity | AuROC |
|---|---|---|---|---|
| Imaging data | 85.2% [84.4%, 86.0%] | 66.0% [64.5%, 67.7%] | 93.0% [91.1%, 90.4%] | 0.80 [0.79, 0.81] |
| Clinical data | 83.8% [83.0%, 84.6%] | 70.9% [68.8%, 73.0%] | 89.8% [89.1%, 90.4%] | 0.83 [0.81, 0.84] |
| Combined | 88.0% [87.6%, 88.5%] | 74.3% [73.0%, 75.6%] | 94.6% [93.9%, 95.3%] | 0.86 [0.85, 0.87] |
| imaging and | | | | |
| clinical data | | | | |

Numbers in brackets are 95% confidence intervals.

E.8.c Classifying Liver Stiffness Using Clinical Data Alone

Using only clinical data, the model classified patients with an AuROC of 0.83 (Table 2), achieving a significantly greater AuROC (p=0.003) compared to the one using only imaging data. The accuracy of this model was 83.8%, the sensitivity was 70.9%, and the specificity was 89.8%.

E.8.d Classifying Liver Stiffness Using Both Imaging and Clinical Data

The DeepLiverNet combining both T2-weighted MR imaging and clinical data was able to correctly classify patients with an AuROC of 0.86 (Table 2). This was significantly greater than imaging data alone (p<0.0001) or clinical data alone (p<0.0001). The DeepLiverNet model achieved an accuracy of 88.0%, with a sensitivity of 74.3% and specificity of 94.6%.

E.8.e External Validation

Ninety-five MRI examinations from 95 unique patients were included in our external validation experiment. Fifty-nine patients with a mean liver stiffness <3 kPa had a mean age of 15.0 (4.7) years; 40/59 (67.8%) patients were male. Thirty-six patients with a mean liver stiffness ≥3 kPa had a mean age of 14.1 (4.7) years; 28/36 (77.8%) patients were male. There was no significant difference in age (p=0.45) or sex (p=0.30) between groups. Patients with a mean liver stiffness <3 kPa had a mean liver stiffness of 2.3 (0.3) kPa, while patients with a mean liver stiffness ≥3 kPa had a mean liver stiffness of 4.4 (1.4) kPa. Ninety (94.7%) MRE examinations were performed on 1.5T MRI scanners, and only 5 (5.3%) MRE examinations were performed on 3T MRI scanners.

The trained DeepLiverNet for classifying liver stiffness using both clinical and imaging features was able to correctly classify patients with an AuROC of 0.77. This model achieved an accuracy of 80.0%, with a sensitivity of 61.1% and specificity of 91.5%. Using the imaging data alone, the model had an accuracy of 77.2%, sensitivity of 60.3%, specificity of 89.4%, and AuROC of 0.75. With the clinical data alone, the model achieved an accuracy of 75.0%, sensitivity of 60.9%, specificity of 87.3%, and AuROC of 0.74.

E.8.f Visualization of Discriminative Image Regions

The most discriminative image regions ranked by our DeepLiverNet for a given T2-weighted liver image were visualized using gradient-weighted class activation mapping (Grad-CAM) technique²¹³ in FIG. 15. Coarse location heat maps were overlaid with the input liver images. FIG. 4 demonstrates axial T2-weighted liver images (FIGS. 9A-C, left column) and their most discriminative regions (FIGS. 9A-C, right column) from three subjects with different liver stiffness values (ranging from 1.4 kPa to 6.9 kPa). Subjective assessment of maps commonly showed localization to the left hepatic lobe and medial portion of the spleen as well as intervening tissues (e.g., gastrohepatic ligament region).

E.8.g Ranking of Clinical Features

We applied a connection weights algorithm²¹⁴ to rank the importance of clinical and non-deep imaging features. The 10 most discriminative features for classifying liver stiffness in our DeepLiverNet model included total bilirubin, fibrosis-4 score, gamma-glutamyl transferase, direct bilirubin, MRI liver volume, MRI chemical shift-encoded fat fraction, aspartate aminotransferase to platelet ratio index (APRI), body mass index, aspartate aminotransferase, and serum albumin.

E.9 Discussion

Deep learning, which simultaneously learns data representation and decision making, is a state-of-the-art artificial intelligence technique, and it has achieved exceptional performance in numerous fields, such as image recognition, object detection, and natural language processing.²⁰¹ We focused on supervised deep learning, where a model is given a set of input data (e.g., clinical data and/or MR images) as well as associated labels (i.e., liver stiffness) to learn the latent relationship between input data and labels. To the best of our knowledge, this is the first study that developed a deep learning model to predict categorical liver stiffness in pediatric and young adult patients by using clinical features and traditional anatomic MR images. In this retrospective study, DeepLiverNet was proposed and evaluated for a liver stiffening classification task. By integrating clinical and T2-weighted MRI liver data, DeepLiverNet achieved an AuROC of 0.86 and an accuracy of 88.1% at internal validation. This model reached a slightly lower AuROC of 0.77 and an accuracy of 80.0% at external validation on an independent cross-platform patient cohort. This multi-channel deep learning model outperformed the single-channel models trained with either clinical or imaging data alone. Such a model with continued refinement could be used to reliably identify patients with normal liver stiffness at point of care (e.g., integrated within the MR console) to triage the need for additional MRE testing, and thus potentially avoid MRE in up to two-thirds of candidate patients, shortening examination length, and lowering healthcare costs.

Overfitting is a phenomenon that occurs when a model fits the training data closely, but has difficulty being generalized to additional unseen datasets. It is especially common when classifying medical images, where the heterogeneity of biologic processes is inherent and training samples are relatively limited. Thus, two strategies were applied to mitigate the model overfitting. The first strategy was transfer learning. Pretrained ImageNet models²⁰⁷⁻²¹⁰ that were trained on ˜1.2 million non-medical color images (dogs, cats, cars, etc.) were reused to help the training of the DeepLiverNet on medical images (i.e., anatomic T2-weighted MR images) in a liver stiffening classification task. Although there are differences between non-medical color images and gray-scale medical images in terms of image content, basic image elements such as edges, shapes, and blobs are similar across any image. After comparing various ImageNet models, we opted to use VGG-19 in our work. VGG-19 model achieved the best performance in our optimization experiments, even though it has relatively simpler architecture than other models (i.e., Inception, ResNet, and NASNet). The architecture design of deep learning models depends on the complexity of the task.²¹⁵ While those deeper models are useful for a general computer vision classification task with a thousand categories, they may not be optimal to be reused in our 2-way classification task. Indeed, a similar trend has been reported previously.²⁰² The other strategy used for minimizing the possibility of model overfitting was data augmentation. Image augmentation methods have been applied frequently to enlarge variability of training samples and enhance generalizability of models.^{207,212,216} With these two strategies, our internal and external validation results show promise for our DeepLiverNet as an off-the-shelf product in the near-future for clinic use.

Visualization of the imaging channel of DeepLiverNet could explicitly demonstrate from where DeepLiverNet extracted image features for liver stiffness stratification. Although the exemplary model utilizes entire T2-weighted images for prediction, it is noted that similar regions covering the left hepatic lobe and medial spleen were identified on saliency maps for correctly classifying patients, despite their varying degrees of liver stiffening. A bold interpretation of the resulting Grad-CAM heat maps may be that the exemplary deep learning model was emphasizing the relationship between liver and spleen (and intervening tissues, such as the gastrohepatic ligament), such as the ratio of liver/spleen volumes. It has been established that the morphology of the left hepatic lobe and spleen change with progressive liver fibrosis and cirrhosis.²¹⁷ In our previous study, liver volume was also recognized as a predictor of liver stiffening by a support vector machine learning model.

By deciphering the clinical channel of DeepLiverNet, the most discriminative clinical features were revealed for classifying liver stiffness. These features (including total bilirubin, fibrosis-4 score, gamma-glutamyl transferase, direct bilirubin, MRI liver volume, etc.) are quite similar to those identified from an overlapping subject cohort that used more traditional machine learning (support vector machine) to identify clinical and imaging features predictive of liver stiffness. Based on existing literature, changes in such clinical features have been associated with progressive chronic liver disease and increasing liver fibrosis/cirrhosis.

In the current embodiment, only four axial T2-weighted liver images, from where the liver stiffness values were assessed in the MRE examinations, were used in the model evaluation. It is conceivable that additional T2-weighted slices or even the whole liver could be harnessed to leverage the model performance. In the current embodiment, only T2-weighted fat-suppressed liver images were used for the DeepLiverNet. Additional imaging data from other pulse sequences, such as T1-weighted or diffusion-weighted imaging, may improve model performance. Similar deep learning methodologies may be used to predict liver stiffness on a continuous scale and categorically (or continuously based on advanced digital pathology) stage liver fibrosis on a histopathologic basis.

E.10 Conclusion

In conclusion, a deep learning model incorporating clinical features and T2-weighted MR images has demonstrated a means of classifying patients into normal/minimally elevated versus moderately/severely elevated liver stiffness with an accuracy up to 88%. Both internal and external validation experiments were performed using data on MRI scanners from two different manufacturers from subjects with a variety of chronic liver diseases. This model may be used as the foundation for predicting liver histologic fibrosis, perhaps eliminating the need for biopsy in some patients with suspected or known chronic liver disease.

### F. Example Computing Environments

The current disclosure provides methods and systems for diagnosing liver disease. The computing engines, modules, machine learning modules, machine learning engines, deep learning modules/engines, training systems, architectures and other disclosed functions are embodied as computer instructions that may be installed for running on one or more computer devices and/or computer servers. In some instances, a local user can connect directly to the system; in other instances, a remote user can connect to the system via a network.

Example networks can include one or more types of communication networks. For example communication networks can include (without limitation), the Internet, a local area network (LAN), a wide area network (WAN), various types of telephone networks, and other suitable mobile or cellular network technologies, or any combination thereof. Communication within the network can be realized through any suitable connection (including wired or wireless) and communication technology or standard (wireless fidelity (WiFi®), 4G, 5G, long-term evolution (LTE™)), and the like as the standards develop.

The computer device(s) and/or computer server(s) can be configured with one or more computer processors and a computer memory (including transitory computer memory and/or non-transitory computer memory), configured to perform various data processing operations. The computer device(s) and/or computer server(s) also include a network communication interface to connect to the network(s) and other suitable electronic components.

Example local and/or remote user devices can include a personal computer, portable computer, smartphone, tablet, notepad, dedicated server computer devices, any type of communication device, and/or other suitable compute devices.

The computer device(s) and/or computer server(s) can include one or more computer processors and computer memories (including transitory computer memory and/or non-transitory computer memory), which are configured to perform various data processing and communication operations associated with diagnosing liver disease as disclosed herein based upon information obtained/provided (such as the MRI data, MRE data, clinical data, etc. discussed above) over the network, from a user and/or from a storage device. In some implementations, storage device can be physically integrated to the computer device(s) and/or computer server(s); in other implementations, storage device can be a repository such as a Network-Attached Storage (NAS) device, an array of hard-disks, a storage server or other suitable repository separate from the computer device(s) and/or computer server(s).

In some instances, storage device can include the machine-learning models/engines and other software engines or modules as described herein. Storage device can also include sets of computer executable instructions to perform some or all the operations described herein.

### REFERENCES

The current disclosure cites the following references by numeric notation. The disclosures of each of these references are incorporated by reference.
1. Centers for Disease Control and Prevention. Accessed on Dec. 6, 2019. Retrieved from https://www.cdc.gov/nchs/pressroom/sosmap/liver disease mortality/liver disease.htm.
2. Younossi Z M, Stepanova M, Younossi Y, Golabi P, Mishra A, Rafiq N, Henry L. Epidemiology of chronic liver diseases in the USA in the past three decades. Gut 2019. PMID: 31366455.
3. Younossi Z M, Golabi P, de Avila L, Paik J M, Srishord M, Fukui N, Qiu Y, Burns L, Afendy A, Nader F. The global epidemiology of NAFLD and NASH in patients with type 2 diabetes: A systematic review and meta-analysis. J Hepatol 2019; 71:793-801. PMID: 31279902.
4. Ko J S. New Perspectives in Pediatric Nonalcoholic Fatty Liver Disease: Epidemiology, Genetics, Diagnosis, and Natural History. Pediatr Gastroenterol Hepatol Nutr 2019; 22:501-510. PMID: 31777715; PMCID: PMC6856496.
5. Younossi Z M, Blissett D, Blissett R, Henry L, Stepanova M, Younossi Y, Racila A, Hunt S, Beckerman R. The economic and clinical burden of nonalcoholic fatty liver disease in the United States and Europe. Hepatology 2016; 64:1577-1586. PMID: 27543837.
6. UNOS. Accessed on Dec. 10, 2019. Retrieved from https://unos.org/data/transplant-trends.
7. Xanthakos S A, Trout A T, Dillman J R. Magnetic resonance elastography assessment of fibrosis in children with NAFLD: Promising but not perfect. Hepatology 2017; 66:1373-1376. PMID: 28741294; PMCID: PMC5650547.
8. Trout A T, Sheridan R M, Serai S D, Xanthakos S A, Su W, Zhang B, Wallihan D B. Diagnostic Performance of MR Elastography for Liver Fibrosis in Children and Young Adults with a Spectrum of Liver Diseases. Radiology 2018; 287:824-832. PMID: 29470938.
9. Dillman J R, Serai S D, Trout A T, Singh R, Tkach J A, Taylor A E, Blaxall B C, Fei L, Miethke A G. Diagnostic performance of quantitative magnetic resonance imaging biomarkers for predicting portal hypertension in children and young adults with autoimmune liver disease. Pediatr Radial 2019; 49:332-341. PMID: 30607435.
10. Dillman J R, Trout A T, Costello E N, Serai S D, Bramlage K S, Kohli R, Xanthakos S A. Quantitative Liver MRI-Biopsy Correlation in Pediatric and Young Adult Patients With Nonalcoholic Fatty Liver Disease: Can One Be Used to Predict the Other? American Journal of Roentgenology 2018; 210:166-174. PMID: WOS:000418427200036.
11. Li H, Parikh N A, Wang J, Merhar S, Chen M, Parikh M, Holland S, He L. Objective and Automated Detection of Diffuse White Matter Abnormality in Preterm Infants Using Deep Convolutional Neural Networks. Front Neurosci 2019; 13:610. PMID: 31275101; PMCID: PMC6591530.
12. Chen M, Li H, Wang J, Dillman J R, Parikh N A, He L. A Multichannel Deep Neural Network Model Analyzing Multiscale Functional Brain Connectome Data for Attention Deficit Hyperactivity Disorder Detection. 2019; 2:e190012.
13. Dillman J R, Heider A, Bilhartz J L, Smith E A, Keshavarzi N, Rubin J M, Lopez M J. Ultrasound shear wave speed measurements correlate with liver fibrosis in children. Pediatric Radiology 2015; 45:1480-1488. PMID: WOS:000360438800007.
14. He L, Li H, Dudley J A, Maloney T C, Brady S L, Somasundaram E, Trout A T, Dillman J R. Machine Learning Prediction of Liver Stiffness Using Clinical and T2-Weighted MRI Radiomic Data. American Journal of Roentgenology 2019; 213:592-601. PMID: 31120779.
15. Li H, Parikh N A, He L. A Novel Transfer Learning Approach to Enhance Deep Neural Network Classification of Brain Functional Connectomes. Frontiers in Neuroscience 2018; 12. PMID: WOS:000439602500001.
16. He L, Li H, Holland S, Yuan W, Altaye M, Parikh N. Early prediction of cognitive deficits in very preterm infants using functional connectome data in an artificial neural network framework. NeuroImage: Clinical 2018; 18:290-297; PMCID: PMC5987842.
17. He L, Chen M, Li H, Wang J, Khandwala V, Woo D, Vagal A. Deep Learning Model to Predict Patent Outcome in ICH using Fluid-Attenuated Inversion Recovery Imaging Data. Radiology Society of North American. Chicago; 2019.
18. Li H, He L, Dudley J, Maloney T, Somasundaram E, Brady S L, Parikh N A, Dillman J R. A Deep Transfer Learning Model for Liver Stiffness Classification using Clinical and T2-Weighted MRI Data. International Society for Magnetic Resonance in Medicine Annual Meeting. Sydney, Australia; 2020.
19. Tapper E B, Lok A S F. Use of Liver Imaging and Biopsy in Clinical Practice. N Engl J Med 2017; 377:2296-2297. PMID: 29211669.
20. Guzelbulut F, Cetinkaya Z A, Sezikli M, Yasar B, Ozkara S, Ovunc A O. AST-platelet ratio index, Forns index and FIB-4 in the prediction of significant fibrosis and cirrhosis in patients with chronic hepatitis C. Turk J Gastroenterol 2011; 22:279-285. PMID: 21805418.
21. Shaheen A A, Myers R P. Diagnostic accuracy of the aspartate aminotransferase-to-platelet ratio index for the prediction of hepatitis C-related fibrosis: a systematic review. Hepatology 2007; 46:912-921. PMID: 17705266.
22. Vallet-Pichard A, Mallet V, Nalpas B, Verkarre V, Nalpas A, Dhalluin-Venier V, Fontaine H, Pol S. FIB-4: an inexpensive and accurate marker of fibrosis in HCV infection. comparison with liver biopsy and fibrotest. Hepatology 2007; 46:32-36. PMID: 17567829.
23. Joshi M, Dillman J R, Singh K, Serai S D, Towbin A J, Xanthakos S, Zhang B, Su W Z, Trout A T. Quantitative MRI of fatty liver disease in a large pediatric cohort: correlation between liver fat fraction, stiffness, volume, and patient-specific factors. Abdominal Radiology 2018; 43:1168-1179. PMID: WOS:000430288000014.
24. Yin M, Glaser K J, Manduca A, Mounajjed T, Malhi H, Simonetta D A, Wang R S, Yang L, Mao S A, Glorioso J M, Elgilani F M, Ward C J, Harris P C, Nyberg S L, Shah V H, Ehman R L. Distinguishing between Hepatic Inflammation and Fibrosis with MR Elastography. Radiology 2017; 284:694-705. PMID: WOS:000408010500008.
25. Lambin P, Rios-Velazquez E, Leijenaar R, Carvalho S, van Stiphout R G, Granton P, Zegers C M, Gillies R, Boellard R, Dekker A, Aerts H J. Radiomics: extracting more information from medical images using advanced feature analysis. Eur J Cancer 2012; 48:441-446. PMID: 22257792; PMCID: PMC4533986.
26. Kumar V, Gu Y, Basu S, Berglund A, Eschrich S A, Schabath M B, Forster K, Aerts H J, Dekker A, Fenstermacher D, Goldgof D B, Hall L O, Lambin P, Balagurunathan Y, Gatenby R A, Gillies R J. Radiomics: the process and the challenges. Magn Reson Imaging 2012; 30:1234-1248. PMID: 22898692; PMCID: PMC3563280.
27. Gillies R J, Kinahan P E, Hricak H. Radiomics: Images Are More than Pictures, They Are Data. Radiology 2016; 278:563-577. PMID: WOS:000377702200031.
28. Parekh V, Jacobs M A. Radiomics: a new application from established techniques. Expert Rev Precis Med Drug Dev 2016; 1:207-226. PMID: 28042608; PMCID: PMC5193485.
29. van Griethuysen J J M, Fedorov A, Parmar C, Hosny A, Aucoin N, Narayan V, Beets-Tan R G H, Fillion-Robin J C, Pieper S, Aerts H. Computational Radiomics System to Decode the Radiographic Phenotype. Cancer Res 2017; 77:e104-e107. PMID: 29092951; PMCID: PMC5672828.
30. Bishop C M. Neural networks for pattern recognition: Oxford university press; 1995.
31. Shin H C, Roth H R, Gao M, Lu L, Xu Z, Nogues I, Yao J, Mollura D, Summers R M. Deep Convolutional Neural Networks for Computer-Aided Detection: CNN Architectures, Dataset Characteristics and Transfer Learning. IEEE Trans Med Imaging 2016; 35:1285-1298. PMID: 26886976; PMCID: PMC4890616.
32. Kooi T, van Ginneken B, Karssemeijer N, den Heeten A. Discriminating solitary cysts from soft tissue lesions in mammography using a pretrained deep convolutional neural network. Med Phys 2017; 44:1017-1027. PMID: 28094850.
33. Samala R K, Chan H P, Hadjiiski L M, Helvie M A, Richter C, Cha K. Evolutionary pruning of transfer learned deep convolutional neural network for breast cancer diagnosis in digital breast tomosynthesis. Phys Med Biol 2018; 63:095005. PMID: 29616660; PMCID: PMC5967610.
34. Samala R K, Chan H P, Hadjiiski L, Helvie M A, Wei J, Cha K. Mass detection in digital breast tomosynthesis: Deep convolutional neural network with transfer learning from mammography. Med Phys 2016; 43:6654. PMID: 27908154; PMCID: PMC5135717.
35. Azizi S, Mousavi P, Yan P, Tahmasebi A, Kwak J T, Xu S, Turkbey B, Choyke P, Pinto P, Wood B, Abolmaesumi P. Transfer learning from RF to B-mode temporal enhanced ultrasound features for prostate cancer detection. Int J Comput Assist Radiol Surg 2017; 12:1111-1121. PMID: 28349507.
36. Zheng J, Miao S, Jane Wang Z, Liao R. Pairwise domain adaptation module for CNN-based 2-D/3-D registration. Journal of Medical Imaging 2018; 5:021204. PMID: 29376104; PMCID: PMC5767648.
37. Wolpert D H. Stacked generalization. Neural networks 1992; 5:241-259.
38. Deng H. Interpreting tree ensembles with intrees. International Journal of Data Science and Analytics 2019; 7:277-287.
39. Wen G, Hou Z, Li H, Li D, Jiang L, Xun E. Ensemble of deep neural networks with probability-based fusion for facial expression recognition. Cognitive Computation 2017; 9:597-610.
40. Qiu X, Zhang L, Ren Y, Suganthan P N, Amaratunga G. Ensemble deep learning for regression and time series forecasting. IEEE symposium on computational intelligence in ensemble learning: IEEE; 2014. p 1-6.
41. Zhou Z, Feng J. Deep forest: Towards an alternative to deep neural networks. arXiv: 1702.08835 v1. 2017.
42. Kontschieder P, Fiterau M, Criminisi A, Rota Bulo S. Deep Neural Decision Forests. Proceedings of the IEEE International Conference on Computer Vision; 2015. p 1467-1475.
43. Hosmer Jr O W, Lemeshow S, Sturdivant R X. Applied logistic regression: John Wiley & Sons; 2013.
44. Ho T K. Random decision forests. 1995. IEEE. p 278-282.
45. Cortes C, Vapnik V. Support-Vector Networks. Machine Learning 1995; 20:273-297. PMID: WOS:A1995RX35400003.
46. Ito K, Mitchell D G, Gabata T, Hussain S M. Expanded gallbladder fossa: simple MR imaging sign of cirrhosis. Radiology 1999; 211:723-726. PMID: 10352597.
47. Ito K, Mitchell D G. Hepatic morphologic changes in cirrhosis: MR imaging findings. Abdom Imaging 2000; 25:456-461. PMID: 10931978.
48. Ito K, Mitchell D G, Gabata T. Enlargement of hilar periportal space: a sign of early cirrhosis at MR imaging. J Magn Reson Imaging 2000; 11:136-140. PMID: 10713945.
49. Ito K, Mitchell D G, Kim M J, Awaya H, Koike S, Matsunaga N. Right posterior hepatic notch sign: a simple diagnostic MR finding of cirrhosis. J Magn Reson Imaging 2003; 18:561-566. PMID: 14579399.
50. Orlhac F, Nioche C, Soussan M, Buvat I. Understanding Changes in Tumor Texture Indices in PET: A Comparison Between Visual Assessment and Index Values in Simulated and Patient Data. J Nucl Med 2017; 58:387-392. PMID: 27754906.
51. Davatzikos C, Rathore S, Bakas S, Pati S, Bergman M, Kalarot R, Sridharan P, Gastounioti A, Jahani N, Cohen E, Akbari H, Tunc B, Doshi J, Parker D, Hsieh M, Sotiras A, Li H, Ou Y, Doot R K, Bilello M, Fan Y, Shinohara R T, Yushkevich P, Verma R, Kontos D. Cancer imaging phenomics toolkit: quantitative imaging analytics for precision diagnostics and predictive modeling of clinical outcome. J Med Imaging (Bellingham) 2018; 5:011018. PMID: 29340286; PMCID: PMC5764116.
52. Yu Y, Wang J, Ng C W, Ma Y, Mo S, Fong E L S, Xing J, Song Z, Xie Y, Si K, Wee A, Welsch R E, So P T C, Yu H. Deep learning enables automated scoring of liver fibrosis stages. Sci Rep 2018; 8:16016. PMID: 30375454; PMCID: PMC6207665.
53. Choi K J, Jang J K, Lee S S, Sung Y S, Shim W H, Kim H S, Yun J, Choi J Y, Lee Y, Kang B K, Kim J H, Kim S Y, Yu E S. Development and Validation of a Deep Learning System for Staging Liver Fibrosis by Using Contrast Agent-enhanced CT Images in the Liver. Radiology 2018; 289:688-697. PMID: 30179104.
54. Dillman J R, Tkach J A, Gandi D, Singh R, Miethke A G, Jayaswal A, Trout A T. Relationship between Magnetic Resonance Imaging Spleen T1 Relaxation and Other Radiologic and Clinical Biomarkers of Liver Fibrosis in Children and Young Adults with Autoimmune Liver Disease. Abdominal Radiology 2020; Under Review.
55. Prior F, Almeida J, Kathiravelu P, Kurc T, Smith K, Fitzgerald T, Saltz J. Open access image repositories: high-quality data to enable machine learning research. Clinical Radiology 2019: (Epub).
56. Sahiner B, Pezeshk A, Hadjiiski L M, Wang X, Drukker K, Cha K H, Summers R M, Giger M L. Deep learning in medical imaging and radiation therapy. Medical physics 2019; 46:e1-e36.
57. Tang A, Tam R, Cadrin-Chenevert A, Guest W, Chong J, Barfett J, Chepelev L, Cairns R, Mitchell J R, Cicero M D. Canadian Association of Radiologists white paper on artificial intelligence in radiology. Canadian Association of Radiologists Journal 2018; 69:120-135.
58. LeCun Y, Bengio Y, Hinton G. Deep learning. Nature 2015; 521:436-444. PMID: 26017442.
59. Weiss K, Khoshgoftaar™, Wang D. A survey of transfer learning. Journal of Big data
60. Pan S J, Yang Q. A survey on transfer learning. IEEE Transactions on knowledge and data engineering 2010; 22:1345-1359.
61. Krizhevsky A, Sutskever I, Hinton G E. Imagenet classification with deep convolutional neural networks. Advances in neural information processing systems; 2012. p 1097-1105.
62. Amodei D, Ananthanarayanan S, Anubhai R, Bai J, Battenberg E, Case C, Casper J, Catanzaro B, Cheng Q, Chen G. Deep speech 2: End-to-end speech recognition in english and mandarin. International conference on machine learning; 2016. p 173-182.
63. Hinton G, Deng L, Yu D, Dahl G, Mohamed A-r, Jaitly N, Senior A, Vanhoucke V, Nguyen P, Kingsbury B. Deep neural networks for acoustic modeling in speech recognition. IEEE Signal processing magazine 2012; 29:82-97.
64. Holzinger A, Biemann C, Pattichis C S, Kell D B. What do we need to build explainable AI systems for the medical domain? arXiv preprint arXiv:171209923 2017.
65. Parikh R B, Obermeyer Z, Navathe A S. Regulation of predictive analytics in medicine. Science 2019; 363:810-812. PMID: 30792287; PMCID: PMC6557272.
66. Towards trustable machine learning. Nat Biomed Eng 2018; 2:709-710. PMID: 31015650.
67. Shwartz-Ziv R, Tishby N. Opening the black box of deep neural networks via information. arXiv preprint arXiv:170300810 2017.
68. Papadakis G Z, Karantanas A H, Tsikankis M, Tsatsakis A, Spandidos D A, Marias K. Deep learning opens new horizons in personalized medicine. Biomedical reports 2019; 10:215-217.
69. Samek W, Wiegand T, Muller K-R. Explainable artificial intelligence: Understanding, visualizing and interpreting deep learning models. arXiv preprint arXiv:170808296 2017.
70. Selvaraju R R, Das A, Vedantam R, Cogswell M, Parikh D, Batra D. Grad-CAM: Why did you say that? arXiv preprint arXiv:161107450 2016.
71. Olden J D, Jackson D A. Illuminating the “black box”: a randomization approach for understanding variable contributions in artificial neural networks. Ecological modelling 2002; 154:135-150.
72. Selvaraju R R, Cogswell M, Das A, Vedantam R, Parikh D, Batra D. Grad-cam: Visual explanations from deep networks via gradient-based localization. Proceedings of the IEEE International Conference on Computer Vision; 2017. p 618-626.
73. Zhou B, Khosla A, Lapedriza A, Oliva A, Torralba A. Learning deep features for discriminative localization. Proceedings of the IEEE conference on computer vision and pattern recognition; 2016. p 2921-2929.
74. Simonyan K, Vedaldi A, Zisserman A. Deep inside convolutional networks: Visualising image classification models and saliency maps. arXiv preprint arXiv:13126034 2013.
75. Zeiler M D, Fergus R. Visualizing and understanding convolutional networks. European conference on computer vision: Springer; 2014. p 818-833.
76. Ribeiro M T, Singh S, Guestrin C. Why should i trust you?: Explaining the predictions of any classifier. Proceedings of the 22nd ACM SIGKDD international conference on knowledge discovery and data mining: ACM; 2016. p 1135-1144.
77. Bluemke D A, Moy L, Bredella M A, Ertl-Wagner B B, Fowler K J, Goh V J, Halpern E F, Hess C P, Schiebler M L, Weiss C R. Assessing Radiology Research on Artificial Intelligence: A Brief Guide for Authors, Reviewers, and Readers—From the Radiology Editorial Board. Radiology 2019:192515. PMID: 31891322.
78. He H, Bai Y, Garcia E A, Li S. ADASYN: Adaptive synthetic sampling approach for imbalanced learning. IEEE International Joint Conference on Neural Networks 2008. p 1322-1328.
79. Ronneberger O, Fischer P, Brox T. U-Net: Convolutional Networks for Biomedical Image Segmentation. Medical Image Computing and Computer-Assisted Intervention 2015; 9351:234-241. PMID: WOS:000365963800028.
80. Johnson W E, Li C, Rabinovic A. Adjusting batch effects in microarray expression data using empirical Bayes methods. Biostatistics 2007; 8:118-127.
81. Russakovsky O, Deng J, Su H, Krause J, Satheesh S, Ma S, Huang Z, Karpathy A, Khosla A, Bernstein M. Imagenet large scale visual recognition challenge. International journal of computer vision 2015; 115:211-252.
82. lntraobserver and interobserver variations in liver biopsy interpretation in patients with chronic hepatitis C. The French METAVIR Cooperative Study Group. Hepatology 1994; 20:15-20. PMID: 8020885.
83. Isgro G, Calvaruso V, Andreana L, Luong T V, Garcovich M, Manousou P, Alibrandi A, Maimone S, Marelli L, Davies N, Patch D, Dhillon A P, Burroughs A K. The relationship between transient elastography and histological collagen proportionate area for assessing fibrosis in chronic viral hepatitis. Journal of Gastroenterology 2013; 48:921-929. PMID: WOS:000323284500004.
84. Goldberg D J, Surrey L F, Glatz A C, Dodds K, O'Byrne M L, Lin H C, Fogel M, Rome J J, Rand E B, Russo P, Rychik J. Hepatic Fibrosis Is Universal Following Fontan Operation, and Severity is Associated With Time From Surgery: A Liver Biopsy and Hemodynamic Study. Journal of the American Heart Association 2017; 6. PMID: WOS:000404098600011.
85. Jovicich J, Czanner S, Greve D, Haley E, van der Kouwe A, Gollub R, Kennedy D, Schmitt F, Brown G, MacFall J. Reliability in multi-site structural MRI studies: effects of gradient non-linearity correction on phantom and human data. Neuroimage 2006; 30:436-443.
86. Fortin J-P, Parker D, Tune; B, Watanabe T, Elliott M A, Ruparel K, Roalf D R, Satterthwaite T D, Gur R C, Gur R E. Harmonization of multi-site diffusion tensor imaging data. Neuroimage 2017; 161:149-170.
87. Fortin J-P, Cullen N, Sheline Y I, Taylor W D, Aselcioglu I, Cook P A, Adams P, Cooper C, Fava M, McGrath P J. Harmonization of cortical thickness measurements across scanners and sites. Neuroimage 2018; 167:104-120.
88. Yu M, Linn K A, Cook P A, Phillips M L, McInnis M, Fava M, Trivedi M H, Weissman M M, Shinohara R T, Sheline Y I. Statistical harmonization corrects site effects in functional connectivity measurements from multi-site fMRI data. Human brain mapping 2018; 39:4213-4227.
89. Deng J, Dong W, Socher R, Li L-J, Li K, Fei-Fei L. Imagenet: A large-scale hierarchical image database. IEEE Conference on Computer Vision and Pattern Recognition: IEEE; 2009. p 248-255.
90. Simonyan K, Zisserman A. Very deep convolutional networks for large-scale image recognition. arXiv preprint arXiv:14091556 2014.
91. He K, Zhang X, Ren S, Sun J. Deep residual learning for image recognition. Proceedings of the IEEE conference on computer vision and pattern recognition; 2016. p 770-778.
92. He K, Zhang X, Ren S, Sun J. Identity mappings in deep residual networks. European conference on computer vision: Springer; 2016. p 630-645.
93. Xie S, Girshick R B, Dollar P, Tu Z, He K. Aggregated residual transformations for deep neural networks. The IEEE Conference on Computer Vision and Pattern Recognition; 2017. p 1492-1500.
94. Szegedy C, Vanhoucke V, loffe S, Shlens J, Wojna Z. Rethinking the inception architecture for computer vision. Proceedings of the IEEE conference on computer vision and pattern recognition; 2016. p 2818-2826.
95. Szegedy C, loffe S, Vanhoucke V, Alemi A. lnception-v4, inception-resnet and the impact of residual connections on learning. CoRR abs/1602.07261. arXiv preprint arXiv:160207261v2 2016.
96. landola F, Moskewicz M, Karayev S, Girshick R, Darrell T, Keutzer K. Densenet: Implementing efficient convnet descriptor pyramids. arXiv preprint arXiv:14041869 2014.
97. Zoph B, Vasudevan V, Shlens J, Le Q V. Learning transferable architectures for scalable image recognition. Proceedings of the IEEE conference on computer vision and pattern recognition; 2018. p 8697-8710.
98. Bengio Y. Learning Deep Architectures for AI. Found Trends Mach Learn 2009; 2:1-127.
99. Liu H, Simonyan K, Vinyals O, Fernando C, Kavukcuoglu K. Hierarchical representations for efficient architecture search. arXiv preprint arXiv:171100436 2017.
100. Zoph B, Le Q V. Neural architecture search with reinforcement learning. arXiv preprint arXiv:161101578 2016.
101. Real E, Moore S, Selle A, Saxena S, Suematsu Y L, Tan J, Le Q V, Kurakin A. Large-scale evolution of image classifiers. Proceedings of the 34th International Conference on Machine Learning. Volume 70; 2017. p 2902-2911.
102. Luo R, Tian F, Qin T, Chen E, Liu T-Y. Neural architecture optimization. Advances in neural information processing systems; 2018. p 7816-7827.
103. Wong K C, Moradi M. SegNAS3D: Network Architecture Search with Derivative-Free Global Optimization for 3D Image Segmentation. 2019. Springer. p 393-401.
104. Liu H, Simonyan K, Yang Y. Darts: Differentiable architecture search. arXiv preprint arXiv:180609055 2018.
105. Johnson R, Zhang T. Accelerating stochastic gradient descent using predictive variance reduction. Advances in neural information processing systems; 2013. p 315-323.
106. Kingma D P, Ba J. Adam: A method for stochastic optimization. arXiv preprint arXiv:14126980 2014.
107. Tieleman T, Hinton G. Lecture 6.5-rmsprop: Divide the gradient by a running average of its recent magnitude. COURSERA: Neural networks for machine learning 2012; 4:26-31.
108. Ouchi J, Hazan E, Singer Y. Adaptive subgradient methods for online learning and stochastic optimization. Journal of Machine Learning Research 2011; 12:2121-2159.
109. Glorot X, Bengio Y. Understanding the difficulty of training deep feedforward neural networks. 2010. p 249-256.
110. Lemaitre G, Nogueira F, Aridas C K. Imbalanced-learn: A python toolbox to tackle the curse of imbalanced datasets in machine learning. The Journal of Machine Learning Research 2017; 18:559-563.
111. Haixiang G, Yijing L, Shang J, Mingyun G, Yuanyue H, Bing G. Learning from class-imbalanced data: Review of methods and applications. Expert Systems with Applications 2017; 73:220-239.
112. Krawczyk B. Learning from imbalanced data: open challenges and future directions. Progress in Artificial Intelligence 2016; 5:221-232.
113. Hu S, Liang Y, Ma L, He Y. MSMOTE: improving classification performance when training data is imbalanced. The Second International Workshop on Computer Science and Engineering. Volume 2: IEEE; 2009. p 13-17.
114. Chollet F. Building powerful image classification models using very little data. Keras Blog 2016.
115. Yasaka K, Akai H, Kunimatsu A, Abe O, Kiryu S. Liver Fibrosis: Deep Convolutional Neural Network for Staging by Using Gadoxetic Acid-enhanced Hepatobiliary Phase MR Images. Radiology 2018; 287:146-155. PMID: 29239710.
116. Parekh V S, Jacobs M A. Deep learning and radiomics in precision medicine. Expert Rev Precis Med Drug Dev 2019; 4:59-72. PMID: 31080889; PMCID: PMC6508888.
117. Sun W Q, Zheng B, Qian W. Automatic feature learning using multichannel ROI based on deep structured algorithms for computerized lung cancer diagnosis. Computers in Biology and Medicine 2017; 89:530-539. PMID: WOS:000413376600051.
118. Li Z J, Wang Y Y, Yu J H, Guo Y, Cao W. Deep Learning based Radiomics {DLR) and its usage in noninvasive IDH1 prediction for low grade glioma. Scientific Reports 2017; 7. PMID: WOS:000405464200104.
119. Kontos D, Summers R M, Giger M. Special Section Guest Editorial: Radiomics and Deep Learning. J Med Imaging (Bellingham) 2017; 4:041301. PMID: 29322066; PMCID: PMC5752704.
120. Arimura H, Soufi M, Kamezawa H, Ninomiya K, Yamada M. Radiomics with artificial intelligence for precision medicine in radiation therapy. J Radiat Res 2019; 60:150-157. PMID: 30247662; PMCID: PMC6373667.
121. Linguraru M G, Sandberg J K, Jones E C, Summers R M. Assessing splenomegaly: automated volumetric analysis of the spleen. Acad Radiol 2013; 20:675-684. PMID: 23535191; PMCID: PMC3945039.
122. Liu J Q, Huo Y K, Xu Z B, Assad A, Abramson R G, Landman B A. Multi-Atlas Spleen Segmentation on CT Using Adaptive Context Learning. Medical Imaging 2017: Image Processing 2017; 10133. PMID: WOS:000405564600007.
123. Xu Z B, Burke R P, Lee C P, Baucom R B, Poulose B K, Abramson R G, Landman B A. Efficient multi-atlas abdominal segmentation on clinically acquired CT with SIMPLE context learning. Medical Image Analysis 2015; 24:18-27. PMID: WOS:000360252700002.
124. Gibson E, Giganti F, Hu Y, Bonmati E, Bandula S, Gurusamy K, Davidson B, Pereira S P, Clarkson M J, Barratt D C. Automatic Multi-Organ Segmentation on Abdominal CT With Dense V-Networks. IEEE Trans Med Imaging 2018; 37:1822-1834. PMID: 29994628; PMCID: PMC6076994.
125. Gruber N, Antholzer S, Jaschke W, Kremser C, Haltmeier MJapa. A Joint Deep Learning Approach for Automated Liver and Tumor Segmentation. 2019.
126. Huo Y K, Liu J Q, Xu Z B, Harrigan R L, Assad A, Abramson R G, Landman B A. Robust Multicontrast MRI Spleen Segmentation for Splenomegaly Using Multi-Atlas Segmentation. Ieee Transactions on Biomedical Engineering 2018; 65:336-343. PMID: WOS:000422914700010.
127. Bobo Alf, Bao S X, Huo Y K, Yao Y, Virostko J, Plassard A J, Lyu I, Assad A, Abramson R G, Hilmes M A, Landman B A. Fully Convolutional Neural Networks Improve Abdominal Organ Segmentation. Medical Imaging 2018: Image Processing 2018; 10574. PMID: WOS:000435027500098.
128. Wang K, Mamidipalli A, Retson T, Bahrami N, Hasenstab K, Blansit K, Bass E, Delgado T, Cunha G, Middleton MSJRAI. Automated CT and MRI Liver Segmentation and Biometry Using a Generalized Convolutional Neural Network. 2019; 1:180022.
129. Xu Z, Lee C P, Heinrich M P, Modat M, Rueckert D, Ourselin S, Abramson R G, Landman B A. Evaluation of Six Registration Methods for the Human Abdomen on Clinically Acquired CT. IEEE Trans Biomed Eng 2016; 63:1563-1572. PMID: 27254856; PMCID: PMC4972188.
130. Huo Y, Liu J, Xu Z, Harrigan R L, Assad A, Abramson R G, Landman B A. Multi-atlas Segmentation Enables Robust Multi-contrast MRI Spleen Segmentation for Splenomegaly. Proc SPIE Int Soc Opt Eng 2017; 10133. PMID: 28649156; PMCID: PMC5480961.
131. Zhou X R, Takayama R, Wang S, Zhou X X, Hara T, Fujita H. Automated segmentation of 3D anatomical structures on CT images by using a deep convolutional network based on end-to-end learning approach. Medical Imaging 2017: Image Processing 2017; 10133. PMID: WOS:000405564600072.
132. Aerts H J. The Potential of Radiomic-Based Phenotyping in Precision Medicine: A Review. JAMA Oncol 2016; 2:1636-1642. PMID: 27541161.
133. Aerts HJWL, Velazquez E R, Leijenaar R T H, Parmar C, Grossmann P, Cavalho S, Bussink J, Monshouwer R, Haibe-Kains B, Rietveld D, Hoebers F, Rietbergen M M, Leemans C R, Dekker A, Quackenbush J, Gillies R J, Lambin P. Decoding tumour phenotype by noninvasive imaging using a quantitative radiomics approach. Nature Communications 2014; 5. PMID: WOS:000338836200003.
134. Marusyk A, Almendro V, Polyak K. Intra-tumour heterogeneity: a looking glass for cancer? Nat Rev Cancer 2012; 12:323-334. PMID: 22513401.
135. Yip S S, Aerts H J. Applications and limitations of radiomics. Phys Med Biol 2016; 61:R150-166. PMID: 27269645; PMCID: PMC4927328.
136. Fedorov A, Beichel R, Kalpathy-Cramer J, Finet J, Fillion-Robin J C, Pujol S, Bauer C, Jennings D, Fennessy F, Sonka M, Buatti J, Aylward S, Miller J V, Pieper S, Kikinis R. 3D Slicer as an image computing platform for the Quantitative Imaging Network. Magnetic Resonance Imaging 2012; 30:1323-1341. PMID: WOS:000309946000013.
137. Parmar C, Velazquez E R, Leijenaar R, Jermoumi M, Carvalho S, Mak R H, Mitra S, Shankar B U, Kikinis R, Haibe-Kains B, Lambin P, Aerts HJWL. Robust Radiomics Feature Quantification Using Semiautomatic Volumetric Segmentation. Plos One 2014; 9. PMID: WOS:000339992400042.
138. Ulyanov D, Vedaldi A, Lempitsky VJapa. Instance normalization: The missing ingredient for fast stylization. 2016.
139. Maas A L, Hannun A Y, Ng A Y. Rectifier nonlinearities improve neural network acoustic models. The 30th International Conference on Machine Learning. Volume 30; 2013. p 3.
140. Drozdzal M, Vorontsov E, Chartrand G, Kadoury S, Pal C. The Importance of Skip Connections in Biomedical Image Segmentation. Deep Learning and Data Labeling for Medical Applications 2016; 10008:179-187. PMID: WOS:000389936900019.
141. Kim W R, Brown R S, Jr., Terrault N A, EI-Serag H. Burden of liver disease in the United States: summary of a workshop. Hepatology 2002; 36:227-242. PMID: 12085369.
142. Bataller R, Brenner D A. Liver fibrosis. J Clin Invest 2005; 115:209-218. PMID: 15690074; PMCID: PMC546435.
143. Brancatelli G, Federle M P, Ambrosini R, Lagalla R, Carriero A, Midiri M, Vilgrain V. Cirrhosis: CT and MR imaging evaluation. Eur J Radiol 2007; 61:57-69. PMID: 17145154.
144. Rockey D C, Caldwell S H, Goodman Z D, Nelson R C, Smith A D. Liver biopsy. Hepatology 2009; 49:1017-1044. PMID: 19243014.
145. Bedossa P, Carrat F. Liver biopsy: the best, not the gold standard. J Hepatol 2009; 50:1-3. PMID: 19017551.
146. Petitclerc L, Gilbert G, Nguyen B N, Tang A. Liver Fibrosis Quantification by Magnetic Resonance Imaging. Top Magn Reson Imaging 2017; 26:229-241. PMID: 28858038; PMCID: PMC5708719.
147. Petitclerc L, Sebastiani G, Gilbert G, Cloutier G, Tang A. Liver fibrosis: Review of current imaging and MRI quantification techniques. J Magn Reson Imaging 2017; 45:1276-1295. PMID: 27981751.
148. Smith A D, Porter K K, Elkassem A A, Sanyal R, Lockhart M E. Current Imaging Techniques for Noninvasive Staging of Hepatic Fibrosis. AJR Am J Roentgenol 2019:1-13. PMID: 30973773.
149. Rustogi R, Horowitz J, Harmath C, Wang Y, Chalian H, Ganger D R, Chen Z E, Bolster B O, Jr., Shah S, Miller F H. Accuracy of MR elastography and anatomic MR imaging features in the diagnosis of severe hepatic fibrosis and cirrhosis. J Magn Reson Imaging 2012; 35:1356-1364. PMID: 22246952; PMCID: PMC3495186.
150. Kudo M, Zheng R Q, Kim S R, Okabe Y, Osaki Y, Iijima H, Itani T, Kasugai H, Kanematsu M, Ito K, Usuki N, Shimamatsu K, Kage M, Kojiro M. Diagnostic accuracy of imaging for liver cirrhosis compared to histologically proven liver cirrhosis. A multicenter collaborative study. Intervirology 2008; 51 Suppl 1:17-26. PMID: 18544944.
151. Bahl G, Cruite I, Wolfson T, Gamst A C, Collins J M, Chavez A D, Barakat F, Hassanein T, Sirlin C B. Noninvasive classification of hepatic fibrosis based on texture parameters from double contrast-enhanced magnetic resonance images. J Magn Reson Imaging 2012; 36:1154-1161. PMID: 22851409; PMCID: PMC4803477.
152. House M J, Bangma S J, Thomas M, Gan E K, Ayonrinde O T, Adams L A, Olynyk J K, St Pierre T G. Texture-based classification of liver fibrosis using MRI. J Magn Reson Imaging 2015; 41:322-328. PMID: 24347292.
153. Hagan M, Asrani S K, Talwalkar J. Non-invasive assessment of liver fibrosis and prognosis. Expert Rev Gastroenterol Hepatol 2015; 9:1251-1260. PMID: 26377444.
154. Mahmoud-Ghoneim D, Amin A, Corr P J R, Oncology. MRI-based texture analysis: a potential technique to assess protectors against induced-liver fibrosis in rats. 2009; 43:30-40.
155. Yu H, Buch K, Li B, O'Brien M, Soto J, Jara H, Anderson S W. Utility of texture analysis for quantifying hepatic fibrosis on proton density MRI. J Magn Reson Imaging 2015; 42:1259-1265. PMID: 26477447.
156. Sandrasegaran K, Akisik F M, Lin C, Tahir B, Raj an J, Saxena R, Aisen A M. Value of diffusion-weighted MRI for assessing liver fibrosis and cirrhosis. AJR Am J Roentgenol 2009; 193:1556-1560. PMID: 19933647.
157. Ozkurt H, Keskiner F, Karatag O, Alkim C, Erturk S M, Basak M. Diffusion Weighted MRI for Hepatic Fibrosis: Impact of b-Value. Iran J Radiol 2014; 11:e3555. PMID: 24693297; PMCID: PMC3955853.
158. Cassinotto C, Feldis M, Vergniol J, Mouries A, Cochet H, Lapuyade B, Hocquelet A, Juanola E, Foucher J, Laurent F, De Ledinghen V. MR relaxometry in chronic liver diseases: Comparison of T1 mapping, T2 mapping, and diffusion-weighted imaging for assessing cirrhosis diagnosis and severity. Eur J Radiol 2015; 84:1459-1465. PMID: 26032126.
159. Taouli B, Tolia A J, Losada M, Babb J S, Chan E S, Bannan M A, Tobias H. Diffusion-weighted MRI for quantification of liver fibrosis: Preliminary experience. American Journal of Roentgenology 2007; 189:799-806. PMID: WOS:000249595800010.
160. Freiman M, Sela Y, Edrei Y, Pappo O, Joskowicz L, Abramovitch R. Multi-class SVM model for fMRI-based classification and grading of liver fibrosis. Medical Imaging 2010: Computer-Aided Diagnosis 2010; 7624. PMID: WOS:000284752400026.
161. lmajo K, Kessoku T, Honda Y, Tomeno W, Ogawa Y, Mawatari H, Fujita K, Yoneda M, Taguri M, Hyogo H, Sumida Y, Ono M, Eguchi Y, Inoue T, Yamanaka T, Wada K, Saito S, Nakajima A. Magnetic Resonance Imaging More Accurately Classifies Steatosis and Fibrosis in Patients With Nonalcoholic Fatty Liver Disease Than Transient Elastography. Gastroenterology 2016; 150:626-+. PMID: WOS:000370648100024.
162. Yin M, Talwalkar J A, Glaser K J, Manduca A, Grimm R C, Rossman P J, Fidler J L, Ehman R L. Assessment of hepatic fibrosis with magnetic resonance elastography. Clinical Gastroenterology and Hepatology 2007; 5:1207-1213. PMID: WOS:000250363600017.
163. Sela Y, Freiman M, Dery E, Edrei Y, Safadi R, Pappo O, Joskowicz L, Abramovitch R. fMRI-Based Hierarchical SVM Model for the Classification and Grading of Liver Fibrosis. Ieee Transactions on Biomedical Engineering 2011; 58:2574-2581. PMID: WOS:000294127700017.
164. Polikar R. Ensemble based systems in decision making. IEEE Circuits and systems magazine 2006; 6:21-45.
165. Dietterich T G. Ensemble methods in machine learning. International workshop on multiple classifier systems: Springer; 2000. p 1-15.
166. Kaggle. https://www.kaggle.com/. Accessed on Dec. 20, 2019.
167. Yin M, Glaser K J, Talwalkar J A, Chen J, Manduca A, Ehman R L. Hepatic MR Elastography: Clinical Performance in a Series of 1377 Consecutive Examinations. Radiology 2016; 278:114-124. PMID: 26162026; PMCID: PMC4688072.
168. Furlan A, Tublin M E, Yu L, Chopra K B, Lippello A, Behari J. Comparison of 20 Shear Wave Elastography, Transient Elastography, and MR Elastography for the Diagnosis of Fibrosis in Patients With Nonalcoholic Fatty Liver Disease. AJR Am J Roentgenol 2020; 214:W20-w26. PMID: 31714842.
169. Güneş F, Wolfinger R, Tan P-Y. Stacked ensemble models for improved prediction accuracy. Proceedings of Statistical Annual Symposium; 2017. p 1-19.
170. Dietterich T G. Ensemble learning. The handbook of brain theory and neural networks 2002; 2:110-125.
171. Hoerl A E, Kennard RWJT. Ridge regression: Biased estimation for nonorthogonal problems. 1970; 12:55-67.
172. Tibshirani R. Regression shrinkage and selection via the lasso. Journal of the Royal Statistical Society Series B (Methodological) 1996:267-288.
173. Shi Y, Xia F, Li Q J, Li J H, Yu B, Li Y, An H, Glaser K J, Tao S Z, Ehman R L, Guo Q Y. Magnetic Resonance Elastography for the Evaluation of Liver Fibrosis in Chronic Hepatitis B and C by Using Both Gradient-Recalled Echo and Spin-Echo Echo Planar Imaging: A Prospective Study. American Journal of Gastroenterology 2016; 111:823-833. PMID: WOS:000382006300020.
174. Serai S D, Towbin A J, Podberesky D J. Pediatric liver MR elastography. Digestive diseases and sciences 2012; 57:2713-2719.
175. Muthupillai R, Lomas D, Rossman P, Greenleaf J F, Manduca A, Ehman R L. Magnetic resonance elastography by direct visualization of propagating acoustic strain waves. science 1995; 269:1854-1857.
176. Palmeri M L, Nightingale K R. Acoustic radiation force-based elasticity imaging methods. Interface Focus 2011; 1:553-564. PMID: 22419986; PMCID: PMC3262278.
177. Muthupillai R, Lomas D J, Rossman P J, Greenleaf J F, Manduca A, Ehman R L. Magnetic resonance elastography by direct visualization of propagating acoustic strain waves. Science 1995; 269:1854-1857. PMID: 7569924.
178. Sarvazyan A P, Rudenko O V, Swanson S D, Fowlkes J B, Emelianov S Y. Shear wave elasticity imaging: a new ultrasonic technology of medical diagnostics. Ultrasound Med Biol 1998; 24:1419-1435. PMID: 10385964.
179. Chalasani, N., et al., The diagnosis and management of nonalcoholic fatty liver disease: practice guidance from the American Association for the Study of Liver Diseases. Hepatology, 2018. 67(1): p. 328 357.
180. Lavanchy, D., The global burden of hepatitis C. Liver international, 2009. 29: p. 74 81.
181. Tapper, E. B. and A. S. F. Lok, Use of liver imaging and biopsy in clinical practice. New England Journal of Medicine, 2017. 377(8): p. 756 768.
182. Serai, S. D., et al., Putting it all together: established and emerging MRI techniques for detecting and measuring liver fibrosis. Pediatric radiology, 2018. 48(9): p. 1256 1272.
183. Smith, A. D., et al., Current Imaging Techniques for Noninvasive Staging of Hepatic Fibrosis. American Journal of Roentgenology, 2019: p. 1 13.
184. Banerjee, R., et al., Multiparametric magnetic resonance for the non invasive diagnosis of liver disease. Journal of hepatology, 2014. 60(1): p. 69 77.
185. Dillman, J. R., et al., Ultrasound shear wave speed measurements correlate with liver fibrosis in children. Pediatric radiology, 2015. 45(10): p. 1480 1488.
186. Yin, M., et al., Hepatic MR elastography: clinical performance in a series of 1377 consecutive examinations. Radiology, 2015. 278(1): p. 114 124.
187. Shi, Y., et al., MR elastography for the assessment of hepatic fibrosis in patients with chronic hepatitis B infection: does histologic necroinflammation influence the measurement of hepatic stiffness? Radiology, 2014. 273(1): p. 88 98.
188. Joshi, M., et al., Quantitative MRI of fatty liver disease in a large pediatric cohort: correlation between liver fat fraction, stiffness, volume, and patient specific factors. Abdominal Radiology, 2018. 43(5): p. 1168 1179.
189. DiPaola, F. W., et al., Effect of Fontan operation on liver stiffness in children with single ventricle physiology. European radiology, 2017. 27(6): p. 2434 2442.
190. Rotemberg, V., et al., The impact of hepatic pressurization on liver shear wave speed estimates in constrained versus unconstrained conditions. Physics in Medicine & Biology, 2011. 57(2): p. 329.
191. Trout, A. T., et al., Diagnostic performance of MR elastography for liver fibrosis in children and young adults with a spectrum of liver diseases. Radiology, 2018. 287(3): p. 824 832.
192. Serai, S. D., A. J. Towbin, and D. J. Podberesky, Pediatric liver MR elastography. Digestive diseases and sciences, 2012. 57(10): p. 2713 2719.
193. Muthupillai, R., et al., Magnetic resonance elastography by direct visualization of propagating acoustic strain waves. science, 1995. 269(5232): p. 1854 1857.
194. Bahl, M., et al., High Risk Breast Lesions: A Machine Learning Model to Predict Pathologic Upgrade and Reduce Unnecessary Surgical Excision. Radiology, 2018. 286(3): p. 810 818.
195. Dawes, T. J. W., et al., Machine Learning of Three dimensional Right Ventricular Motion Enables Outcome Prediction in Pulmonary Hypertension: A Cardiac MR Imaging Study. Radiology, 2017. 283(2): p. 381 390.
196. Kickingereder, P., et al., Radiogenomics of Glioblastoma: Machine Learning based Classification of Molecular Characteristics by Using Multiparametric and Multiregional MR Imaging Features. Radiology, 2016. 281(3): p. 907 918.
197. Wu, H., et al., Classifier Model Based on Machine Learning Algorithms: Application to Differential Diagnosis of Suspicious Thyroid Nodules via Sonography. AJR Am J Roentgenol, 2016: p. 1 6.
198. Abajian, A., et al., Predicting Treatment Response to Intra arterial Therapies for Hepatocellular Carcinoma with the Use of Supervised Machine Learning An Artificial Intelligence Concept. JVasc Intery Radiol, 2018. 29(6): p. 850 857 e1.
199. Kline, T. L., et al., Performance of an Artificial Multi observer Deep Neural Network for Fully Automated Segmentation of Polycystic Kidneys. J Digit Imaging, 2017. 30(4): p. 442 448.
200. Mutasa, S., et al., MABAL: a Novel Deep Learning Architecture for Machine Assisted Bone Age Labeling. J Digit Imaging, 2018.
201. LeCun, Y., Y. Bengio, and G. Hinton, Deep learning. Nature, 2015. 521(7553): p. 436 44.
202. Lakhani, P. and B. Sundaram, Deep learning at chest radiography: automated classification of pulmonary tuberculosis by using convolutional neural networks. Radiology, 2017. 284(2): p. 574 582.
203. Serai, S. D., J. R. Dillman, and A. T. Trout, Spin echo echo planar imaging MR elastography versus gradient echo MR elastography for assessment of liver stiffness in children and young adults suspected of having liver disease. Radiology, 2016. 282(3): p. 761 770.
204. He, L., et al., Machine Learning Prediction of Liver Stiffness Using Clinical and T2 Weighted MRI Radiomic Data. American Journal of Roentgenology, 2019: p. 1 10.
205. Sawh, M. C., et al., Normal range for MR elastography measured liver stiffness in children without liver disease. Journal of Magnetic Resonance Imaging, 2019. Epub ahead of print.
206. Yin, M., et al., Assessment of hepatic fibrosis with magnetic resonance elastography. Clinical Gastroenterology and Hepatology, 2007. 5(10): p. 1207 1213. e2.
207. Simonyan, K. and A. Zisserman, Very deep convolutional networks for large scale image recognition. arXiv preprint arXiv:1409.1556, 2014.
208. He, K., et al. Deep residual learning for image recognition. inProceedings of the IEEE conference on computer vision and pattern recognition. 2016.
209. Szegedy, C., et al. Rethinking the inception architecture for computer vision. in Proceedings of the IEEE conference on computer vision and pattern recognition. 2016.
210. Zoph, B., et al. Learning transferable architectures for scalable image recognition. in Proceedings of the IEEE conference on computer vision and pattern recognition. 2018.
211. Kingma, D. P. and J. Ba, Adam: A method for stochastic optimization. arXiv preprint arXiv:1412.6980, 2014. 212. Krizhevsky, A., I. Sutskever, and G. E. Hinton, Imagenet classification with deep convolutional neural networks. Advances in Neural Information Processing Systems, 2012.
213. Selvaraju, R. R., et al., Grad CAM: Why did you say that? arXiv preprint arXiv:1611.07450, 2016.
214. Olden, J. D. and D. A. Jackson, Illuminating the “black box”: a randomization approach for understanding variable contributions in artificial neural networks. Ecological modelling, 2002. 154(1 2): p. 135 150.
215. Andrew, N., Machine learning yearning. 2017.
216. Szegedy, C., et al. Going deeper with convolutions. in Proceedings of the IEEE conference on computer vision and pattern recognition. 2015.
217. Pickhardt, P. J., et al., Hepatosplenic volumetric assessment at MDCT for staging liver fibrosis. European radiology, 2017. 27(7): p. 3060 3068.

What is claimed is:

1. A method for performing a medical diagnosis of liver disease comprising the steps of:
receiving multiparametric MRI data and clinical data;
diagnosing aspects of liver disease by applying one or more machine learning models to the MRI data and clinical data, wherein the one or more machine learning models uses biopsy-derived histologic data as a reference standard; and
communicating detected and quantified liver disease aspect information to a user.

2. The method of claim 1, wherein the one or more machine learning models extracts and integrates radiomic features and deep features from the multiparametric MRI data in the diagnosing step.

3. The method of claim 2, wherein the multiparametric MRI data represents segmented portions of the liver and spleen.

4. The method of claim 3, wherein the diagnosing step utilizes a convolutional neural network provided with both Short and Long Residual connections (SLRes-U-Net) to simultaneously take multiparametric MRI as inputs and jointly segment the liver.

5. The method of claim 2, wherein the radiomic features comprise constructs capturing spatial appearance and spectral properties of tissues through imaging descriptors of grey-scale signal intensity distribution, shape morphology, and inter-voxel signal intensity pattern.

6. The method of claim 2, wherein the deep features comprise complex abstractions of patterns learned from input images through multiple non-linear transformations estimated by data driven deep transfer learning training.

7. The method of claim 1, wherein:
the receiving step receives MRE data; and
the diagnosing step diagnoses liver disease by applying at least one machine learning model to the multiparametric MRI data, MRE data and clinical data.

8. The method of claim 7, wherein the diagnosing step predicts biopsy-derived liver fibrosis stage and liver fibrosis percentage.

9. The method of claim 7, wherein the clinical data comprises demographic data, diagnosis data and laboratory testing data.

10. The method of claim 1, wherein the diagnosis step predicts MRE-derived shear liver stiffness utilizing a deep learning regression model on at least the multiparametric MRI data.

11. The method of claim 1, further comprising a step of training at least one of the machine learning models using transfer learning.

12. The method of claim 1, further comprising a step of integrating at least one of the machine learning models using ensemble learning.

13. The method of claim 1, wherein at least one of the machine learning models of the diagnosing step segments liver and spleen using a convolutional neural network provided with both short and long residual connections to extract radiomic and deep features from the multiparametric MRI data.

14. The method of claim 13, wherein the diagnosing step further implements data augmentation as part of the liver and spleen segmenting process.

15. A system for performing a medical diagnosis of liver disease comprising:
one or more sources of multiparametric MRI data and clinical data;
a machine learning engine configured to receive the multiparametric MRI data and clinical data and diagnosing aspects of liver disease by applying one or more machine learning models to the multiparametric MRI data and clinical data; and
a computerized output communicating detected and quantified liver disease aspect information from the machine learning engine to a user.

16. The system of claim 15, wherein the machine learning engine extracts and integrates radiomic features and deep features from the multiparametric MRI data in the diagnosing step.

17. The system of claim 16, wherein the multiparametric MRI data represents segmented portions of the liver.

18. The system of claim 17, wherein the machine learning engine comprises a convolutional neural network provided with both short and long residual connections to simultaneously take multiparametric MRI as inputs and jointly segment the liver and spleen.

19. The method of claim 16, wherein the radiomic features comprise constructs capturing spatial appearance and spectral properties of tissues through imaging descriptors of grey-scale signal intensity distribution, shape morphology, and inter-voxel signal intensity pattern.

20. The method of claim 16, wherein the deep features comprise complex abstractions of patterns learned from input images through multiple non-linear transformations estimated by data driven deep transfer learning training.

21. The system of claim 15, wherein:
the one or more sources include MRE data; and
the machine learning engine is configured to diagnoses liver disease by applying the one or more machine learning models to the multiparametric MRI data, MRE data and clinical data.

22. The system of claim 21, wherein the machine learning engine is configured to predict biopsy-derived liver fibrosis stage and liver fibrosis percentage.

23. The system of claim 21, wherein the clinical data comprises demographic data, diagnosis data and laboratory testing data.

24. The system of claim 15, wherein the machine learning engine is configured to predict MRE-derived shear liver stiffness utilizing a deep learning regression model on at least the MRI data.

25. The system of claim 15, wherein at least one of the one or more machine learning models is integrated using transfer learning.

26. The system of claim 15, wherein at least one of the one or more machine learning models is trained using ensemble learning.

27. The system of claim 15, wherein the machine learning engine comprises a convolutional neural network provided with both short and long residual connections to extract radiomic and deep features from the multiparametric MRI data to segment the liver and spleen.

28. The system of claim 27, wherein the machine learning engine implements data augmentation as part of the liver segmenting process.

29. The system of claim 27, wherein the machine learning engine includes a u-shaped convolutional neural network provided with both short and long residual connections to simultaneously take multiparametric MRI data as input to jointly segment the liver and spleen.

30. The system of claim 29, wherein the convolutional neural network includes a symmetric architecture, having an encoder that extracts spatial features from the multiparametric MRI data, and a decoder that constructs a segmentation map.

31. The system of claim 29, wherein the convolutional neural network includes a 3-dimensional convolutional block and a 3-dimensional residual block.

32. The system of claim 31, wherein the convolutional 3-dimensional convolutional block includes a 3-dimensional convolution layer, an instance normalization layer and a leaky rectified linear unit later.

33. The system of claim 31, wherein the 3-dimensional residual block includes an additional short residual connection, linking input with output feature maps of the residual block and performing a summation operation.

34. The system of claim 31, wherein the convolutional neural network includes an encoder that extracts spatial features from the MRI data, the encoder including a sequence of 3-dimensional convolutional blocks and a 3-dimensional residual blocks.

35. The system of claim 34, wherein the sequence is followed by a down-sampling operation that is repeated multiple times, and after the down sampling operation at each level, the number of features channels is doubled.

36. The system of claim 35, wherein the convolutional neural network includes a decoder that constructs a segmentation map, the decoder including a succession of 3-dimensional convolutional blocks and 3-dimensional residual blocks, which up-sample feature maps and reduce the number of feature channels by half at each successive level.

37. A method for performing a medical diagnosis of the liver comprising the steps of:
receiving multiparametric MRI data, MRE data and clinical data concerning a patient's liver;
applying a plurality of machine learning models to the multiparametric MRI data, MRE data and clinical data;
combining the plurality of machine learning models into an ensemble deep learning model;
diagnosing aspects of liver disease based upon an output of the ensemble deep learning model; and
communicating liver disease aspect information to a user.

38. The method of claim 37, wherein the combining step includes a step of identifying, for each of the plurality of machine learning models, each model's predictive feature identification process by applying deep learning feature ranking and saliency map approaches.

39. A system for performing a medical diagnosis of the liver comprising
a deep learning framework segmenting liver and spleen image information using a convolutional neural network with both short and long residual connections to extract radiomic and deep features from multiparametric MRI; and
an ensemble deep learning model quantifying liver fibrosis stage and percentage using the integration the extracted radiomic and deep features, MRE data, and clinical data.

40. The system of claim 39, further comprising a deep learning model quantifying MRE-derived liver stiffness using the extracted radiomic and deep features and routinely-available clinical data.

41. The system of claim 39, further comprising a feature ranking module revealing the model's predictive feature identification process by applying deep learning feature ranking and saliency map approaches.
